(19) United States
(12) Patent Application Publication
(10) Pub. No.: US 2024/0254258 A1
(43) Pub. Date: Aug. 1, 2024

(54) ANTI-ALK ANTIBODIES AND USES THEREOF

(71) Applicant: Trellis Bioscience, Inc., Redwood City, CA (US)

(72) Inventors: Angeles Estelles, Redwood City, CA (US); Mikhail Gishizky, Redwood City, CA (US); Stefan Ryser, Redwood City, CA (US); Lawrence Kauvar, Redwood City, CA (US)

(73) Assignee: Trellis Bioscience, Inc., Redwood City, CA (US)

(21) Appl. No.: 18/545,533

(22) Filed: Dec. 19, 2023

Related U.S. Application Data
(63) Continuation of application No. PCT/US2022/035151, filed on Jun. 27, 2022.
(60) Provisional application No. 63/215,947, filed on Jun. 28, 2021.

(51) Int. Cl. C07K 16/40; A61K 9/16; A61K 39/00; A61K 47/68

(52) U.S. Cl. CPC C07K 16/40; A61K 9/1617; A61K 47/6817; A61K 47/6871; A61K 2039/505; C07K 2317/24; C07K 2317/31; C07K 2317/565; C07K 2317/622

(57) ABSTRACT

Disclosed herein are monoclonal antibodies, and antigen-binding fragments thereof, that immunospecifically bind to human Anaplastic Lymphoma Kinase (ALK). Also provided are methods of use thereof, such the treatment of cancer. Also included are antibody-drug conjugates (ADCs) including the monoclonal antibodies and/or antigen-binding fragments thereof, linked to an effector agent, such as a cytotoxic agent. Also provided are method of using such ADCs. Nucleic acid molecules, vectors, and host cells for the monoclonal antibodies and antigen-binding fragments thereof are also provided, as are pharmaceutical and veterinarian compositions including the monoclonal antibodies, and antigen-binding fragments thereof, and/or the associated ADCs.

### CROSS-REFERENCE TO RELATED APPLICATIONS

This application is a continuation of and claims priority to Int'l Pat. App. PCT/US2022/035151, filed Jun. 27, 2022, titled “ANTI-ALK ANTIBODIES & USES THEREOF,” which claims priority to U.S. Provisional Application No. 63/215,947, filed Jun. 28, 2021, titled “NATIVE HUMAN ANTI-ALK ANTIBODIES.” The entire disclosure of each priority application is hereby fully incorporated herein by reference in its entirety.

### REFERENCE TO SEQUENCE LISTING

This application incorporates by reference in its entirety the sequence listing material in the St.26 XML sequence listing named “12774.141US1” created on Mar. 26, 2024 and containing 58,986 bytes.

### FIELD OF THE INVENTION

The present invention relates to agents that target Tumor Associated Antigens (TAAs), more specifically, it relates to antibodies capable of inducing tumor cell killing by targeting Anaplastic Lymphoma Kinase (ALK). Further, the invention relates to pharmaceutical use of such agents as well as to methods of producing and manufacturing the same.

### BACKGROUND

The classical approach of targeted therapies against cancer over the past 20 years has focused on proteins associated with tumor cells (also known as Tumor Associated Antigens or TAAs). Several such anti-tumor antibody drugs have been commercialized, including those targeting VEGF, Her2, EGFR, and CD20. ALK (Anaplastic Lymphoma Kinase) is a Receptor Tyrosine Kinase that is normally expressed in the developing nervous system (Iwahara, 1997). It belongs to the Insulin receptor family and has been implicated as an oncogene in hematopoietic (anaplastic large cell lymphoma Morris, 1994), as well as non-hematopoietic malignancies: neuroblastoma (Osajima, 2005 and Carpenter, 2012), lung cancer (Wang, 2011; Li, 2011; Guerin, 2015 and Jiang 2016), thyroid cancer (Murugan, 2011), glioblastoma (Powers, 2002) and rhabdomyosarcoma (van Gaal, 2012). Gene amplification resulting in ALK overexpression as well as activating mutations in the tyrosine domain of ALK have been described that link to cellular processes related to oncogenesis, such as cell-cycle progression, survival and cell migration and are a major cause of hereditary neuroblastoma and other cancers (Lovisa, 2015; Tartari, 2008 and Schulte, 2011). Because increased tyrosine kinase activity is induced by such mutations (Wang, 2011), small-molecule kinase inhibitors have been developed, including the marketed drugs Crizotinib (Kwak, 2010), Ceritinib (Marsilje, 2013) and Alectinib (Sakamoto, 2011). However, the approach of treating cancer patients that way has been challenging due to the appearance of drug resistance.

Alternatively, ALK is an appropriate TAA for antibody targeted treatment since its expression is mainly restricted to the tumor, thus minimizing the risk of cytotoxicity in normal tissues. Of interest are ALK antibodies that are able to mediate antibody-dependent cell-mediated cytotoxicity (ADCC). Also of interest are antibodies capable of being internalized in the cancer cells, making them candidates for Antibody Drug Conjugates (ADC) that can carry toxins or radioligands into the cell. Native human antibodies with anti-ALK activity of either type are of particular value for minimizing off-target reactivity and rejection as a foreign protein.

### SUMMARY OF THE INVENTION

In certain example aspects, provided is an isolated monoclonal antibody (mAb), or antigen binding fragment thereof, that specifically binds Anaplastic Lymphoma Kinase (ALK). Such mAbs include TRL10001, TRL10005, TRL10006, TRL10014, or TRL10051, or antigen binding fragment thereof (or variants thereof).

In certain example aspects, the mAb or antigen binding fragment thereof includes a heavy chain variable region (VH) as set forth in the amino acid sequence corresponding to SEQ ID NO:1 and a light chain variable (VL) region as set forth in the amino acid sequence corresponding SEQ ID NO: 8.

In certain example aspects, provided is an isolated monoclonal antibody (mAb) or antigen binding fragment thereof for use in targeting a cancer cell, the mAb or antigen binding fragment thereof comprising a heavy chain variable region of TRL10001 (as set forth in the amino acid sequence corresponding to SEQ ID NO:1) and a light chain variable region of TRL10001 (as set forth in the amino acid sequence corresponding SEQ ID NO: 8).

In certain example aspects, the VH region and VL of the mAb or antigen binding fragment thereof regions share 80%, 85%, 90%, 95%, 96%, 97%, 98%, or 99% sequence identity to the amino acid sequences set forth as SEQ ID NO: 1 and SEQ ID NO: 8, respectively.

In still further example aspects, the isolated mAb or antigen binding fragment thereof that binds ALK includes a heavy chain variable region and a light chain variable region, where the VH region includes complementarity determining regions CDR-H1 (SEQ ID NO: 2), CDR-H2 (SEQ ID NO: 3), and CDR-H3 (SEQ ID NO: 4), while the VL region includes CDR-L1 (SEQ ID NO: 9), CDR-L2 (SEQ ID NO: 10), and CDR-L3 (SEQ ID NO: 11). Alternatively, in certain additional example aspects, the VH region includes CDR-H1 (SEQ ID NO: 5), CDR-H2 (SEQ ID NO: 6), and CDR-H3 (SEQ ID NO: 7), while the VL region includes CDR-L1 (SEQ ID NO: 12), CDR-L2 (SEQ ID NO: 13), and CDR-L3 (SEQ ID NO: 14).

In certain example aspects, the isolated mAb or antigen binding fragment thereof provided herein is monospecific, bispecific, or multispecific. Further, in certain example aspects the mAb or antigen binding fragment thereof is a chimeric antibody or chimeric antigen binding fragment thereof, a human antibody or human antigen binding fragment thereof, a humanized antibody or humanized antigen binding fragment thereof, or a single chain antibody.

In certain example aspects, the antigen binding fragment is an Fv fragment, an Fab fragment, an Fab′ fragment, or an F(ab′)₂ fragment. For example, the Fv fragment can be a single-chain Fv (scFv) fragment, while in certain example aspects the Fab fragment is a single-chain Fab (scFab) fragment. Alternatively, in certain example aspects the isolated mAb is a complete antibody.

In certain example aspects, the mAb or antigen binding fragment thereof targets a pathological cell, such as a cancer cell, thereby inducing an immune response to the pathological target cell. The response, for example, can be phagocytosis or lysis by natural killer lymphocytes. In certain example aspects, the anti-ALK mAb or antigen binding fragment thereof is conjugated to an effector agent to from an immunoconjugate. The effector agent, for example, can be a toxin, radioligand, or other molecule. In certain example aspects, the immunoconjugate can be internalized into the cell, such as a cancer cell, thereby targeting the cancer cell with the effector agent conjugated to the anti-ALK mAb or antigen binding fragment thereof. Such aspects can also be used to treat a subject having cancer with the immunoconjugate.

In still further example aspects, provided is a pharmaceutical or veterinary composition, the composition including the isolated mAb or antigen binding fragment thereof described above or the immunoconjugate thereof. In certain example aspects, the isolated mAb or antigen binding fragment thereof described herein, the immunoconjugate thereof, and/or the composition thereof, is administered to treat a subject, such as a subject having cancer.

Also provided are nucleic acid molecules encoding the mAbs or antigen binding fragments thereof, vectors comprising the nucleic acid molecules, and cells transformed to express the mAbs or antigen binding fragments thereof. Also provided are nucleic acids for use in producing or manufacturing the antibodies/fragments herein, including for use in vivo or in situ.

These and other aspects, objects, features and advantages of the example embodiments will become apparent to those having ordinary skill in the art upon consideration of the following detailed description of illustrated example embodiments.

### BRIEF DESCRIPTION OF THE DRAWINGS

FIG. 1 is a graph showing binding of TRL10001 to ALK expressed at the cell surface of neuroblastoma cell lines, in accordance with certain example embodiments. As shown, binding is proportional to level of expression (highest for NB1 cells, lowest for 1643 cells). Detection by a labelled secondary anti-human antibody is dependent on the human mAb binding to the cells.

FIG. 2 is a graph showing that TRL10001 is internalized after binding to NB1 neuroblastoma cells, in accordance with certain example embodiments. As shown, the cell surface bound, fluorescently labelled TRL10001 is >80% internalized by the cells within 30 minutes. Controls shown include an isotype matched mAb that does not bind to the cells and one that does not become internalized.

### DETAILED DESCRIPTION OF THE INVENTION

The following description of the invention is provided as an enabling teaching of the invention in its best, currently known aspect. Those skilled in the relevant art will recognize that many changes can be made to the aspects described, while still obtaining the beneficial results of the present invention. It will also be apparent that some of the desired benefits of the present invention can be obtained by selecting some of the features of the present invention without utilizing other features. Accordingly, those who work in the art will recognize that many modifications and adaptations to the present invention are possible and can even be desirable in certain circumstances and are a part of the present invention. Thus, the following description is provided as illustrative of the principles of the present invention and not in limitation thereof.

### SUMMARY OF TERMS

As used herein, the singular forms “a,” “an” and “the” include plural referents unless the context clearly dictates otherwise.

Example abbreviations relevant to the present disclosure include: (C) constant, (CH) constant heavy, (CL) constant light, (CDR) complementarity determining region, (Fab) fragment antigen binding, (F(ab′)₂) Fab with additional amino acids, including cysteines necessary for disulfide bonds, (FR) framework region, (Fv) fragment variable, (scFv) single change fragment variable, single-chain Fab (scFab), (H) heavy, (Ig) immunoglobulin, (L) light, (V) variable, (VH) variable heavy, and (VL) variable light. Unless otherwise specified, the mAbs and the antigen binding fragments thereof are collectively referred to herein as “antibody molecules.”

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Additionally, as used herein, relative terms, such as “substantially” and “generally,” and the like are used herein to represent an inherent degree of uncertainty that can be attributed to any quantitative comparison, value, measurement, or other representation. These terms are also utilized herein to represent the degree by which a quantitative representation can vary from a stated reference without resulting in a change in the basic function of the subject matter at issue.

As used herein, “optional” or “optionally” mean that the subsequently described event or circumstance can or cannot occur, and that the description includes instances where said event or circumstance occurs and instances where it does not.

Terms used herein, such as “example,” “exemplary,” or “exemplified,” are not meant to show preference, but rather to explain that the aspect discussed thereafter is merely one example of the aspect presented.

“Administration” or “administering” refers to the introduction of a composition into a subject by a chosen route. For example, if the chosen route is injection, the compositions described herein can be administered by introducing the composition into a target area of a subject, such as a joint, via injection. In certain example embodiments, the compositions described herein can be injected via intra-articular injection. As used herein, “administration” or “administering” also includes systemic delivery, such as the systemic delivery of one or more of the compositions described herein. Similarly, “administering to a subject” or the like indicate a procedure by which the disclosed mAb, fragments thereof, immunoconjugates thereof, and compositions thereof are injected into/provided to a patient such that target cells, tissues, or segments of the body of the subject are contacted with the disclosed human antibody molecules.

Where a nucleic acid molecule is administered, such as for in vivo or in situ expression of an antibody molecule as described herein, such administration generally involves locally or systemically administering an effective amount of nucleic acid molecule and/or pharmaceutical composition thereof to a subject in need thereof, whereby the nucleic acid molecule is transfected (e.g., transiently transfected) into the cells of the subject. The nucleic acid can be transfected into any desired cell such as liver cells (e.g., Kupffer cells and hepatocytes), muscle cells, skeletal cells, lung cells, spleen cells, immune system cells (e.g., mature plasmoblasts, B cells) and combinations of the foregoing. In certain aspects, the nucleic acid can be transfected (transiently transfected) into a secretory cell or an immune system cell.

The term “antibody” and the like are used in a broad sense and include immunoglobulin molecules including, monoclonal antibodies, antibody fragments, bispecific or multispecific antibodies, dimeric, tetrameric or multimeric antibodies, and single chain antibodies. Immunoglobulins can be assigned to five major classes, namely IgA, IgD, IgE, IgG, and igM, depending on the heavy chain constant domain amino acid sequence. IgA and IgG are further sub-classified as the isotypes IgA1, IgA2, IgG1, IgG2, IgG3, and IgG4. Antibody light chains of any vertebrate species can be assigned to one of two clearly distinct types, namely kappa (k) and lambda (l), based on the amino acid sequences of their constant domains.

An “antigen binding fragment” refers to a portion of an immunoglobulin molecule that retains the specific antigen binding properties of the parental full-length antibody (i.e., an antigen binding fragment thereof). Example antibody fragments include heavy chain complementarity determining regions (CDR-H) 1, 2, and 3 and light chain complementarity determining regions (CDR-L) 1, 2, and 3. Other example antibody fragments include a heavy chain variable region (VH) and a light chain variable region (VL). Antibody further fragments include an Fab fragment, a monovalent fragment consisting of the VL, VH, constant light (CL), and constant heavy 1 (CHI) domains; an F(ab)2 fragment, a bivalent fragment comprising two Fab fragments linked by a disulfide bridge at the hinge region; and an Fv fragment consisting of the VL and VH domains of a single arm of an antibody. VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv(scFv) or diabody. See, e.g., Int'l Pat. Pubs. WO 1998/044001, WO1988/001649, WO1994/013804, and WO1992/001047. These antibody fragments are obtained using techniques well known to those of skill in the art, and the fragments can be screened for utility in the same manner as are full length antibodies.

As used herein, “human antibody” refers to an antibody having heavy and light chain variable regions in which both the framework and the antigen binding sites are derived from sequences of human origin. If the antibody contains a constant region, the constant region also is derived from sequences of human origin. A sequence is “derived” from human origin if the variable regions of the antibody are obtained from a system that uses human germline immunoglobulin or rearranged human immunoglobulin genes. Such systems include human immunoglobulin gene libraries displayed on phage, and transgenic non-human animals such as mice or rats carrying human immunoglobulin loci.

In certain example embodiments, a human antibody may contain amino acid differences when compared to the human germline or rearranged immunoglobulin sequences due to, for example, naturally occurring somatic mutations or intentional introduction of substitutions in the framework or antigen binding sites. Typically, a human antibody is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene. In some cases, a human antibody may contain consensus framework sequences derived from human framework sequence analyses, for example as described in Knappik et al., J Mol Biol 296:57-86, 2000, or synthetic HCDR3 incorporated into human immunoglobulin gene libraries displayed on phage, as described in, for example, Shi et al., J Mol Biol 397:385-96, 2010 and Int'l Pat. Pub. No. WO2009/085462.

As those skilled in the art will appreciate, human antibodies, while derived from human immunoglobulin sequences, may be generated using systems such as phage display incorporating synthetic CDRs and/or synthetic frameworks, and/or can be subjected to in vitro mutagenesis to improve antibody properties in the variable regions or the constant regions or both, resulting in antibodies that do not naturally exist within the human antibody germline repertoire in vivo.

As used herein, “chimeric” or “chimeric antibody” refers to an antibody translated from a polynucleotide sequence containing both human and non-human mammal polynucleotide sequences. A “humanized” antibody is one which is produced by a non-human cell or mammal and includes human sequences, e.g., a chimeric antibody. Humanized antibodies are less immunogenic after administration to humans when compared to non-humanized antibodies prepared from another species. In addition, the humanized antibodies of the present invention can be isolated from a knock-in non-human mammal engineered to produce fully human antibody molecules. For example, a humanized antibody may include the human variable region of a chimeric antibody appended to a human constant region to produce a fully human antibody.

As used herein, “monoclonal antibody” refers to a population of antibody molecules of a substantially single molecular composition. A monoclonal antibody composition displays a single binding specificity and affinity for a particular epitope, or in a case of a bispecific monoclonal antibody, a dual binding specificity to two distinct epitopes. “Monoclonal antibody” thus refers to an antibody population with single amino acid composition in each heavy and each light chain, except for possible well-known alterations such as removal of C-terminal lysine from the antibody heavy chain, and processing variations in which there is incomplete cleavage of the N-terminal leader sequence that is produced in the cell and ordinarily cleaved upon secretion. For example, U.S. Pat. No. 8,241,630 describes a commercial antibody in which 5-15% of the antibody population retain the leader sequence. Monoclonal antibodies may have heterogeneous glycosylation within the antibody population. Monoclonal antibody may be monospecific or multispecific, or monovalent, bivalent, or multivalent. A bispecific antibody is included in the term monoclonal antibody.

As used herein, an “epitope” is a portion of an antigen to which an antibody specifically binds. Epitopes usually consist of chemically active (such as polar, non-polar, or hydrophobic) surface groupings of moieties such as amino acids or polysaccharide side chains and can have specific three-dimensional structural characteristics, as well as specific charge characteristics. An epitope can be composed of contiguous and/or discontiguous amino acids that form a conformational spatial unit. For a discontiguous epitope, amino acids from differing portions of the linear sequence of the antigen come in close proximity in 3-dimensional space through the folding of the protein molecule.

The terms “nucleic acid” and “polynucleotide” or variations thereof include any compound or substance that includes a polymer of nucleotides. Each nucleotide is composed of a base, specifically a purine- or pyrimidine base (i.e., cytosine (C), guanine (G), adenine (A), thymine (T) or uracil (U)), a sugar (i.e., deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. The term nucleic acid encompasses deoxyribonucleic acid (DNA) including, e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular.

In addition, the term nucleic acid includes both sense and antisense strands, as well as single stranded and double stranded forms. Moreover, the nucleic acids described herein can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g, cDNA) or RNA (e.g, mRNA) vectors, can be unmodified or modified. For example, mRNA can be chemically modified to enhance the stability of the RNA vector and/or expression of the encoded molecule so that mRNA can be injected into a subject to generate the antibody in vivo (see, e.g., Stadler, C., Bähr-Mahmud, H., Celik, L. et al. Elimination of large tumors in mice by mRNA-encoded bispecific antibodies. Nat Med 23, 815-817 (2017) or EP 2101823 B 1).

The term “vector” refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to generally as “expression vectors.”

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the antibody molecules or immunoconjugates used for the present invention.

A “variant” refers to a polypeptide or a polynucleotide that differs from a reference polypeptide or a reference polynucleotide by one or more modifications for example, substitutions, insertions, or deletions.

The term “binds” or “specifically binds” refers to a non-random binding reaction between two molecules, for example between an antibody or fragment thereof an its antigen. The term “specifically binds” may be used interchangeably with “selectively targets” or “selectively associates.”

As used herein, “treatment,” “treating,” and like terms refer to reducing the severity and/or frequency of symptoms, eliminating symptoms, ameliorating or eliminating the underlying cause of the symptoms, reducing the frequency or likelihood of symptoms and/or their underlying cause, and/or improving or remediating damage caused, directly or indirectly, by the described conditions or disorders. Treating may also include prolonging survival as compared to the expected survival of a subject not receiving the disclosed antibody molecules or pharmaceutical compositions comprising the same.

The terms “prevent,” “preventing” or the like refer to prophylactic or maintenance measures. Subjects for receipt of such prophylactic or maintenance measures include those who are at risk of having the described conditions or disorders due to, for example, genetic predisposition or environmental factors, or those who were previously treated for having the described conditions or disorders and are receiving therapeutically effective doses of the disclosed antibody molecules or pharmaceutical compositions as a maintenance medication.

The phrase “therapeutically effective amount” refers to an amount of the disclosed antibody molecules or pharmaceutical compositions thereof, as described herein, effective to achieve a particular biological or therapeutic or prophylactic result such as biological or therapeutic results disclosed, described, or exemplified herein. The therapeutically effective amount may vary according to factors such as the disease state, age, sex, and weight of the individual, and the ability of the composition to cause a desired response in a subject. Example indicators of a therapeutically effect amount include, for example, improved well-being of the subject, a reduction in tumor size and/or reduction in the number of tumors in a subject, or reduction in cancer cell count. Use of such agents may also delay or prevent emergence of mutated tumors resistant to other agents, such as tyrosine kinase inhibitors.

A used herein, a “pharmaceutically acceptable carrier” or “pharmaceutical acceptable excipient” refers to and includes any material that, when combined with disclosed antibody molecules or compositions thereof, allows the ingredient to retain biological activity and is non-reactive with the subject's immune system. Examples include, but are not limited to, any of the standard pharmaceutical carriers such as a phosphate buffered saline solution, water, and various types of wetting agents (such as polysorbate 20, polysorbate 80, and salts of tris(hydroxymethyl)aminomethane (“Tris”), such as the hydrochloride, acetate, maleate and lactate salts. Stabilizing agents can also be used, such as amino acids (such as histidine, glutamine, glutamate, glycine, arginine), sugars (such as sucrose, glucose, trehalose), chelators (e.g., ETDA), and antioxidants (e.g., reduced cysteine). Preferred diluents for aerosol or parenteral administration are phosphate buffered saline or normal (0.9%) saline. Compositions including such carriers are formulated by well-known conventional methods. E.g., Remington's Pharmaceutical Sciences, by E. W. Martin, Mack Publishing Co., Easton, PA, 19th Edition (1995), for example, describes compositions and formulations suitable for pharmaceutical delivery of the antibody molecules described herein and immunoconjugates thereof.

The terms “purified” or “isolated,” as used herein, refer to biological or synthetic molecules that are removed from their natural environment and are isolated or separated and are free from other components with which they are naturally associated. The term “purified” or “isolated” does not require absolute purity; rather, it is intended as a relative term. Thus, for example, a purified or “substantially pure” antibody preparation is one in which the antibody referred to is more pure than the antibody in its natural environment within a biological system or within a production reaction chamber (as appropriate).

An “effector agent” refers to any molecule or combination of molecules whose activity it is desired to deliver/into and/or localize at cell. Effector agents include, for example, labels, cytotoxins, enzymes, growth factors, transcription factors, antibodies, drugs, etc. The term “label” refers to a detectable compound or composition that is conjugated directly or indirectly to another molecule to facilitate detection of that molecule. Specific, non-limiting examples of labels include fluorescent tags, chemiluminescent tags, haptens, enzymatic linkages, and radioactive isotopes.

The term “immunoconjugate” refers to an antibody molecule attached to one or more effector agents or to a plurality of antibodies attached to one or more effector agents. The term “immunoconjugate” is intended to include effector agents chemically conjugated to the antibody molecules as well as antibody molecules expressed as a fusion protein where the antibody molecule is directly attached or attached through a linker to the effector agents, such as via a peptide linkage or via a ligand (such as biotin) and a high affinity binding domain for that ligand. In certain example embodiments, the immunoconjugate can be formed in vivo, e.g., an antibody molecule as provided herein can be separately administered from the effector agent, with the effector agent and antibody molecule forming the immunoconjugate in vivo (following their administration).

As used herein, a “subject” refers to a vertebrate animal. The vertebrate may be a mammal, for example, such as a human. The subject may be a human “patient.” A subject may be a patient suffering from or suspected of suffering from a disease or condition and may be in need of treatment or diagnosis or may be in need of monitoring for the progression of the disease or condition. The patient may also be in on a treatment therapy that needs to be monitored for efficacy. In some example embodiments, a subject includes a subject suffering from one or more physiological or pathophysiological conditions including cancer or associated with cancer.

### Antibody Molecules

Provided herein are antibodies and antigen binding fragments thereof (collectively “antibody molecules”) that immunospecifically bind to Anaplastic Lymphoma Kinase (ALK). Such antibody molecules can be useful to target or kill cancer cells that display ALK receptor on the cell surface. In certain example embodiments, such antibody molecules are native human antibodies or antigen binding fragments thereof. The mechanism of cell killing could be ADCC, Complement Dependent Cytotoxicity (CDC), or by targeted delivery of a toxin or radioligand (Antibody Drug Conjugate, ADC), including those released inside the cell from an ADC.

U.S. Pat. No. 5,770,421 discloses the full-length protein (“Human ALK Protein Tyrosine Kinase”). U.S. Pat. Nos. 7,902,342 and 8,945,563 disclose antibodies binding to the Extracellular Domain of the receptor tyrosine kinase ALK; these mAbs for treating glioblastoma were generated by two hybrid screening of scFV random libraries. The antibodies disclosed herein, derived from the natural human immune repertoire, are distinct compositions from those prior art antibodies. US2015/0132317 discloses murine mAbs against ALK, which are again distinct from those disclosed herein.

To generate therapeutic antibodies against ALK, the CellSpot™ technology (U.S. Pat. Nos. 7,413,868 and 7,939,344, incorporated herein by reference) was used for identifying rare antibodies (defined by specificity and affinity) within the memory B-cell compartment of the human immune system. Native human anti-ALK antibodies have been discovered by this means. A surprising discovery is that these antibodies have been cloned from healthy blood donors with no known cancer. In other words, the pharmacological approach represented by administration of anti-ALK antibodies appears to have a natural counterpart, consistent with the long-standing immune surveillance concept that in healthy individuals' incipient tumors are eliminated by the immune system. Without wishing to be bound by any particular theory, the low frequency of memory B cells encoding high affinity antibodies to ALK further suggests that this natural mechanism is transient, leaving a footprint in the memory B cell repertoire without leading to long term autoimmune disease.

Human antibodies, such as those disclosed herein, are particularly favorable from both an efficacy perspective (having been cloned from healthy donors) and a safety perspective (reduced chance of off-target reactivity that would create toxicity). The frequency of human antibodies to a particular target in the natural human repertoire is typically orders of magnitude lower than in the repertoire of immunized mice. Accordingly, a high throughput technology capable of surveying millions of individual antibody producing human B lymphocytes is needed. Since human B cells have a very limited lifetime ex vivo (under 10 days), the technology must also operate within that time window.

As those skilled in the art will appreciate, the CellSpot™ technology effectively shrinks an ELISA equivalent assay down to a virtual well of nearly single cell dimensions by capturing secreted IgG from a single cell (˜10 μm in diameter) as a footprint in the vicinity of the cell (˜100 μm in diameter). In that way, 5 million B cells can be readily analyzed at single cell resolution. Further, by use of microscopic multiplexing reagents (combinatorially colored fluorescent latex microspheres, cf. U.S. Pat. No. 6,642,062, incorporated herein by reference), each clone's secreted antibody footprint can be characterized in detail for specificity and/or affinity using multiple biochemical probes. The precision of the quantitative assay is sufficient to enable identification and recovery of extremely rare favorable cells from the survey population. The cloned antibody variable region encoding genes can then be linked to an independently constructed antibody constant region and expressed in an exogenous cell. Such engineered mAbs typically show a phenotype consistent with the native mAb in the original identifying assay.

The antibody molecules provided herein are thus distinct from those found in nature, as they are, in certain example embodiments, prepared recombinantly by constructing nucleic acids that encode a generic form of the constant region of heavy and/or light chain and further encode heterologous variable regions that are representative of human antibodies. Moreover, because in certain example embodiments the B cells are cultured before assay, mutations may arise during this ex vivo period.

In certain example embodiments, the VH and VL regions of the antibody molecules correspond to those as described herein as TRL10001, TRL10005, TRL10006, TRL10014, TRL10051, or variants thereof. For example, provided is an antibody molecule where the VH and VL regions correspond to or consist essentially of the amino acid sequences set forth as SEQ ID NOS: 1 and 8, respectfully (i.e., of TRL10001). In certain example embodiments, the VH region has 85%, 90%, 95%, 96%, 97%, 98%, or 99% sequence identity to the amino acid sequence set forth as SEQ ID NO: 1, while the VL region has 85%, 90%, 95%, 96%, 97%, 98%, or 99% sequence identity to the amino acid sequence set forth as SEQ ID NO: 8. That is, the antibody molecule can be a variant of TRL10001.

In certain example embodiments, provided is an antibody molecule where the VH and VL regions correspond to or consist essentially of the amino acid sequences set forth as SEQ ID NOS: 29 and 31, respectfully (i.e., TRL10005). In certain example embodiments, the VH region has 85%, 90%, 95%, 96%, 97%, 98%, or 99% sequence identity to the amino acid sequence set forth as SEQ ID NO: 29, while the VL region has 85%, 90%, 95%, 96%, 97%, 98%, or 99% sequence identity to the amino acid sequence set forth as SEQ ID NO: 31. That is, the antibody molecule can be a variant of TRL10005.

Further, in certain example embodiments provided is an antibody molecule where the VH and VL regions correspond to or consist essentially of the amino acid sequences set forth as SEQ ID NOS: 33 and 35, respectfully (i.e., TRL10006). In certain example embodiments, the VH region has 85%, 90%, 95%, 96%, 97%, 98%, or 99% sequence identity to the amino acid sequence set forth as SEQ ID NO: 33, while the VL region has 85%, 90%, 95%, 96%, 97%, 98%, or 99% sequence identity to the amino acid sequence set forth as SEQ ID NO: 35. That is, the antibody molecule can be a variant of TRL10006.

In certain example embodiments, provided is an antibody molecule where the VH and VL regions correspond to or consist essentially of the amino acid sequences set forth as SEQ ID NOS: 37 and 39, respectfully (i.e., TRL10014). In certain example embodiments, the VH region has 85%, 90%, 95%, 96%, 97%, 98%, or 99% sequence identity to the amino acid sequence set forth as SEQ ID NO: 37, while the VL region has 85%, 90%, 95%, 96%, 97%, 98%, or 99% sequence identity to the amino acid sequence set forth as SEQ ID NO: 39. That is, the antibody molecule can be a variant of TRL100014.

In certain example embodiments, provided is an antibody molecule where the VH and VL regions correspond to or consist essentially of the amino acid sequences set forth as SEQ ID NOS: 41 and 43, respectfully (i.e., TRL10051). In certain example embodiments, the VH region has 85%, 90%, 95%, 96%, 97%, 98%, or 99% sequence identity to the amino acid sequence set forth as SEQ ID NO: 41, while the VL region has 85%, 90%, 95%, 96%, 97%, 98%, or 99% sequence identity to the amino acid sequence set forth as SEQ ID NO: 43. That is, the antibody molecule can be a variant of TRL10051.

In certain example embodiments, the antibody molecules provided herein include a VH/VL pair of any combination of TRL10001, TRL10005, TRL10006, TRL10014, or TRL10051. That is, any VH or VL domain from TRL10001, TRL10005, TRL10006, TRL10014, or TRL10051 can be interchangeably paired to form the antibody VH/VL portion of the antibody molecules described herein. For example, the VH region of TRL10001 (SEQ ID NO: 1) may be paired with the VL region of TRL10005 (SEQ ID NO: 31), TRL10006 (SEQ ID NO: 35), TRL10014 (SEQ ID NO: 39), or TRL10051 (SEQ ID NO: 43). As another example, the VL region of TRL10001 may be paired with any VH region of TRL10005 (SEQ ID NO: 29), TRL10006 (SEQ ID NO: 33), TRL10014 (SEQ ID NO: 37), or TRL10051 (SEQ ID NO: 51). In certain example embodiments where the VH/VL pairs are interchangeably paired, the amino acid sequence of the VH or VL region may be varied as described herein (i.e., retain a percent identity to the unmodified VH or VL sequence, as described herein). In related example embodiments, either or both of the interchangeable pair can be a variant.

As those skilled in the art will appreciate, antibody variable regions include a set of six complementary determining regions (CDRs) that are responsible for antigen binding. For TRL10001, for example, the CDRs are contained within are contained with the VH/VL regions of SEQ ID NO: 1 and SEQ ID NO: 8, respectively. As those skilled in the art will also, the specificities of the binding of antibodies are defined mostly those of the heavy chain but complemented by those of the light chain as well (the light chains being somewhat interchangeable). Thus, the antibody molecules provided herein may contain the three CDR regions of a heavy chain and optionally the three CDRs of a light chain that matches it. Because binding affinity is also determined by the manner in which the CDRs are arranged on a framework, the antibody molecules provided herein can contain complete variable regions of the heavy chain containing the three relevant CDRs as well as, optionally, the complete light chain variable region comprising the three CDRs associated with the light chain complementing the heavy chain in question. This is true with respect to the antibody molecules that are immunospecific for a single epitope as well as for bispecific antibodies or binding moieties that are able to bind two separate epitopes.

Methods known in the art can be used to identify the CDR regions, including the CDR regions of the heavy chain variable region (i.e., CDR-H1, CDR-H2, and CDR-H3) and those of the light chain variable region (i.e., CDR-L1, CDR-L2, and CDR-L3). Specifically, the most commonly used method for identifying the relevant CDR regions is that of Kabat as disclosed in Wu, T. T., et al., J. Exp. Med. (1970) 132:211-250 and in the book Kabat, E. A., et al. (1983) Sequence of Proteins of Immunological Interest, Bethesda National Institute of Health, 323 pages. Another similar and commonly employed method is that of Chothia, published in Chothia, C., et al., J. Mol. Biol. (1987) 196:901-917 and in Chothia, C., et al., Nature (1989) 342:877-883. An additional modification has been suggested by Abhinandan, K. R., et al., Mol. Immunol. (2008) 45:3832-3839. Another system alternately employed in the art for such definitions is IMGT numbering (Lefranc et al., 2003). The antibody molecules described herein, for example, can include the CDR regions as defined by any one of these systems or other recognized systems known in the art.

For example, using the IMGT system, in certain example embodiments is an antibody molecule that includes CDR amino acid sequences set forth as SEQ ID NO: 2 (CDR-H1), SEQ ID NO: (CDR-H2) 3, and SEQ ID NO: 4 (CDR-H3), as well as SEQ ID NO: 9 (CDR-L1), SEQ ID NO: 10 (CDR-L2), and SEQ ID NO: 11 (CDR-L3). In other example embodiments, such as when using the Kabat system, provided is an antibody molecule that includes CDR amino acid sequences set forth as SEQ ID NO: 5 (CDR-H1), SEQ ID NO: 6 (CDR-H2), and SEQ ID NO: 7 (CDR-H3), as well as SEQ ID NO: 12 (CDR-L1), SEQ ID NO: 13 (CDR-L2), and SEQ ID NO: 14 (CDR-L3). While these IMGT/KABAT CDR regions are for TRL10001, it is contemplated herein that the skilled artisan can, based on the sequences provided herein, identify the CDR regions for TRL10005, TRL10006, TRL10014, and TRL10051.

In certain example embodiments, the amino acid sequences are the CDR sequences arranged on a framework wherein said framework can vary without necessarily affecting specificity or decreasing affinity to an unacceptable level. For example, any identified CDRs of TRL10005, TRL10006, TRL10014, or TRL10051 can be combined with the CDRs identified herein for TRL10001, so long as the binding function of the antibody molecule is maintained. That is, in certain example embodiments, the CDRs of the VH/VL pairs identified above may be mixed to result in different combinations. For example, any of the CDRs of TRL10001 as identified herein can be paired with those of TRL10005, TRL10006, TRL10014, or TRL10051 to result in an antibody to ALK, so long as the affinity for ALK is maintained.

In certain example embodiments, variation to the sequence of the CDRs may be made without impacting, or without substantially impacting, the affinity of the antibody molecule to ALK epitope. That is, one or more specific amino acids may vary, without affecting the antibody molecule's specificity. For example, in certain example embodiments, provided is an antibody having CDRs with 90%, 95%, 96%, 97%, 98%, or 99% sequence identity to the amino acid sequences set forth as SEQ ID NO: 2 (CDR-H1), SEQ ID NO: (CDR-H2) 3, and SEQ ID NO: 4 (CDR-H3), as well as SEQ ID NO: 9 (CDR-L1), SEQ ID NO: 10 (CDR-L2), and SEQ ID NO: 11 (CDR-L3). In certain example embodiments, provided is an antibody having CDRs with 90%, 95%, 96%, 97%, 98%, or 99% sequence identity to the amino acid sequences set forth as SEQ ID NO: 5 (CDR-H1), SEQ ID NO: 6 (CDR-H2), and SEQ ID NO: 7 (CDR-H3), as well as SEQ ID NO: 12 (CDR-L1), SEQ ID NO: 13 (CDR-L2), and SEQ ID NO: 14 (CDR-L3).

In certain example embodiments, bispecific binding moieties may be formed by covalently linking two different binding moieties with different specificities. Multiple technologies now exist for making a single antibody-like molecule that incorporates antigen specificity domains from two separate antibodies (bi-specific antibody). Suitable technologies have been described by MacroGenics (Rockville, MD), Micromet (Bethesda, MD) and Merrimac (Cambridge, MA). See, e.g., Orcutt, K. D., et al., Protein Eng. Des. Sel. (2010) 23:221-228; Fitzgerald, J., et al., MAbs. (2011) 1:3; Baeuerle, P. A., et al., Cancer Res. (2009) 69:4941-4944.

For example, the CDR regions of the heavy and optionally light chain derived from one monospecific mAb may be coupled through any suitable linking means to peptides comprising the CDR regions of the heavy chain sequence and optionally light chain of a second mAb. If the linkage is through an amino acid sequence, the bispecific binding moieties can be produced recombinantly and the nucleic acid encoding the entire bispecific entity expressed recombinantly. As was the case for the antibody molecules with a single specificity, the invention also includes the possibility of binding moieties that bind to one or both of the same epitopes as the bispecific antibody or binding entity/binding moiety that actually contains the CDR regions. The invention further includes bispecific constructs that include the complete heavy and light chain sequences or the complete heavy chain sequence and at least the CDR's of the light chains or the CDR's of the heavy chains and the complete sequence of the light chains.

### Nucleic Acids, Vectors, and Host Cells for Producing Antibody Molecules

The antibody molecules provided herein may be produced using any known techniques in the art. This includes but is not limited to in vitro, in situ, in vivo, or recombinant production. For example, the monoclonal antibodies may be made using the hybridoma method first described by Kohler et al., Nature, 256:495 (1975); see also CURRENT PROTOCOLS IN MOLECULAR BIOLOGY, Ausubel et al. Eds. (Wiley and Sins, New York, N Y 1989 and yearly updates up to and including 2010).

Additionally or alternatively, antibody molecules provided herein may be made by recombinant DNA methods (e.g., U.S. Pat. No. 4,816,567). Thus, provided herein are nucleic acid molecules including nucleotide sequence encoding them, as well as vectors or expression systems that include these nucleotide sequences, cells containing expression systems or vectors for expression of these nucleotide sequences and methods to produce antibody molecules by culturing these cells and recovering the binding molecules.

For example, recombinant expression vectors can include any of the nucleic acid molecules described herein, i.e., nucleic acid molecules encoding any of the VH, VL, and/or CDR sequences as set forth herein. Also, in certain example embodiments provided are host cells or host cell lines into which such vectors have been or can be introduced, as well as methods of producing the antibodies or portions thereof, such as by culturing the host cells (to form a host cell culture) under conditions permitting production of the antibody molecules and recovering the antibody molecules so produced. Any type of cell typically used in recombinant methods can be employed including prokaryotes, yeast, mammalian cells, insect cells and plant cells. Also included are human cells (e.g., muscle cells or lymphocytes) transformed or transfected with a recombinant molecule that encodes the antibody molecules provided herein, including expression in a subject, such as a subject in need of antibody therapy.

Typically, expression systems for the antibody molecules provided herein include a nucleic acid encoding said protein coupled to control sequences for expression. In many embodiments, the control sequences are heterologous to the nucleic acid encoding the protein. Such control sequences include, for example, the human cytomegalovirus (CMV) promoter, with further optimizations in the 5′ and 3′ untranslated regions, polyadenylation signals, and other post-translational response elements. The invention is also directed to nucleic acids encoding the bispecific moieties and to recombinant methods for their production, as described above.

In certain example embodiments, the antibody molecules provided herein include a heavy chain encoded by the polynucleotide sequence set forth as SEQ ID NOS: 15, 30, 34, 38, 36, or 42. In certain example embodiments, the antibody molecules provided herein include a heavy chain encoded by a polynucleotide sequence having 85%, 86%, 87%, 88%, 89%, 90%, 91%, 92%, 93%, 94%, 95%, 96%, 97%, 98%, or 99% sequence identity to the polynucleotide sequence set forth as SEQ ID NOS: 15, 30, 34, 38, or 42.

In certain example embodiments, the antibody molecules provided herein include a light chain encoded by the polynucleotide sequence set forth as SEQ ID NOS: 22, 32, 36, 40, or 44. In certain example embodiments, the antibody molecules provided herein include a heavy chain encoded by a polynucleotide sequence having 85%, 86%, 87%, 88%, 89%, 90%, 91%, 92%, 93%, 94%, 95%, 96%, 97%, 98%, or 99% sequence identity to the polynucleotide sequence set forth as SEQ ID NOS: 22, 32, 36, 40, or 44.

In a preferred embodiment, the antibody molecules provided herein includes a heavy chain encoded by the polynucleotide sequence set forth as SEQ ID NO: 15 a light chain encoded by the polynucleotide sequence set forth as SEQ ID NO: 22. In other example embodiments, the heavy chain is encoded by a polynucleotide sequence having 85%, 86%, 87%, 88%, 89%, 90%, 91%, 92%, 93%, 94%, 95%, 96%, 97%, 98%, or 99% sequence identity to SEQ ID NO: 15, while the light chain is encoded by a polynucleotide sequence having 85%, 86%, 87%, 88%, 89%, 90%, 91%, 92%, 93%, 94%, 95%, 96%, 97%, 98%, or 99% sequence identity to SEQ ID NO: 22.

In certain example embodiments, the CDR-H1, CDR-H2, and CDR-H3 regions of the antibody molecules provided herein are encoded by the polynucleotide sequence set forth in SEQ ID NOS: 16, 17, and 18, respectively. In certain example embodiments, the CDR-H1, CDR-H2, and CDR-H3 regions of the antibody molecules provided herein are encoded by a polynucleotide sequences having 91%, 92%, 93%, 94%, 95%, 96%, 97%, 98%, or 99% sequence identity to the polynucleotide sequences set forth in SEQ ID NOS: 16, 17, and 18, respectively. In certain example embodiments, the CDR-H1, CDR-H2, and CDR-H3 regions of the antibody molecules provided herein are encoded by the polynucleotide sequence set forth in SEQ ID NOS: 19, 20, and 21, respectively. In certain example embodiments, the CDR-H1, CDR-H2, and CDR-H3 regions of the antibody molecules provided herein are encoded by a polynucleotide sequences having 91%, 92%, 93%, 94%, 95%, 96%, 97%, 98%, or 99% sequence identity to the polynucleotide sequences set forth in SEQ ID NOS: 19, 20, and 21, respectively.

In certain example embodiments, the CDR-L1, CDR-L2, and CDR-L3 regions of the antibody molecules provided herein are encoded by the polynucleotide sequence set forth in SEQ ID NOS: 23, 24, and 25, respectively. In certain example embodiments, the CDR-L1, CDR-L2, and CDR-L3 regions of the antibody molecules provided herein are encoded by a polynucleotide sequences having 91%, 92%, 93%, 94%, 95%, 96%, 97%, 98%, or 99% sequence identity to the polynucleotide sequences set forth in SEQ ID NOS: 23, 24, and 25, respectively. In certain example embodiments, the CDR-L1, CDR-L2, and CDR-L3 regions of the antibody molecules provided herein are encoded by the polynucleotide sequence set forth in SEQ ID NOS: 26, 27, and 28, respectively. In certain example embodiments, the CDR-L1, CDR-L2, and CDR-L3 regions of the antibody molecules provided herein are encoded by a polynucleotide sequences having 91%, 92%, 93%, 94%, 95%, 96%, 97%, 98%, or 99% sequence identity to the polynucleotide sequences set forth in SEQ ID NOS: 26, 27, and 28, respectively.

In certain example embodiments, the antibody molecules provided herein can be expressed in vivo or in situ. That is, provided herein is a method for expressing antibody molecules provided herein in vivo or in situ, the method comprising administering to a subject a nucleic acid molecule encoding an antibody molecule as provided herein. When such a nucleic acid molecule including one or more of the nucleic acid sequences provided herein is introduced introduced to a cell of the subject, for example, the cell's machinery then transcribes and/or translates the nucleic acid molecule into the antibody molecule. For example, an mRNA sequence derived from one or more of the cDNA sequences provided herein can be introduced into a cell of the subject, such as via a lipid nanoparticle (LNP) and/or other transfection agents/systems. Once within the cell, the cell's translational machinery can translate the mRNA into the antibody molecule encoded by the introduced mRNA. Such methods and systems of antibody molecule expression, for example, are described in Patel, A. et al., In Vivo Delivery of Nucleic Acid-Encoded Monoclonal Antibodies, BioDrug: 34(3):273-293 (2020), which is hereby incorporated herein in its entirety.

As those skilled in the art will appreciate, a lipid nanoparticle (LNP) typically refers to a particle that includes multiple lipid molecules physically associated with each other by intermolecular forces. LNPs can include, for example, microspheres (including unilamellar and multlamellar vesicles, e.g., liposomes), a dispersed phase in an emulsion, micelles, or an internal phase in a suspension. While a nucleic acid molecule may be encapsulated within an LNP or complexed with an LNP, it is not necessary that the lipid forms liposomes (with aqueous core) only. Some lipid nanoparticles may include a lipid core (e.g., the composition may include a mixture of liposomes and nanoparticles with a lipid core). In such embodiments, the nucleic acid molecules may be encapsulated by LNPs that have an aqueous core and complexed with the LNPs that have a lipid core by non-covalent interactions (e.g., ionic interactions between negatively charged RNA and cationic lipid). Encapsulation and complexation with LNPs can protect RNA from RNase digestion. The encapsulation/complexation efficiency does not have to be 100%. Presence of “naked” RNA molecules (RNA molecules not associated with a liposome) is acceptable. See, e.g., U.S. Pat. App. 2017/0313765 and U.S. Pat. No. 10,087,247, both of which are incorporated herein in their entirety.

As those skilled in art will appreciate, the in vivo or in situ expression of the antibody molecules provided herein can occur via the administration of a single polynucleotide encoding both a VH and VL region or a set of polynucleotides, i.e., one encoding a VH region and one encoding a VL region. Accordingly, provided is a polynucleotide sequence, or set of polynucleotide sequences, the polynucleotide or set of polynucleotide sequences comprising a sequence set forth as SEQ ID NOS: 15 and 22, SEQ ID NOS: 30 and 32, SEQ ID NOS: 34 and 36, or 42 and 44. Such polynucleotides can be used, for example, with any of the methods provided herein where in vivo or in situ expression is desired, such as treating cancer.

### Immunoconjugates

The prototypical anti-ALK antibody described herein specifically binds to, and can be internalized into, a cell expressing Anaplastic Lymphoma Kinase (ALK), such as a cancer cell. As such, the antibody molecules provided herein, as well as polynucleotides encoding the same, can be used alone as therapeutics (e.g., to inhibit growth and/or proliferation of a cancer cell). Additionally or alternatively, the antibody molecules described herein can be coupled to an effector agent to form immunoconjugates, such as antibody-drug conjugates (ADCs), that provide efficient and specific delivery of the effector agent (e.g., cytotoxins, labels, radionuclides, ligands, antibodies, drugs, liposomes, nanoparticles, viral particles, cytokines, and the like) to various cancer cells that express ALK (e.g., isolated cells, metastatic cells, solid tumor cells, etc.).

Immunoconjugates can be formed by conjugating the antibody to an effector agent (e.g., a detectable label, another therapeutic agent, etc.). Suitable agents include, for example, a cytotoxic or cytostatic agent (e.g., a chemotherapeutic agent), a toxin (e.g. an enzymatically active toxin of bacterial, fungal, plant or animal origin, or fragments thereof), and/or a radioactive isotope (i.e., a radioconjugate).

In certain example embodiments, such as those related to treating cancer, cytotoxic agents that can be coupled to the antibody molecules herein include any agent that is detrimental to the growth, viability, or propagation of cells, such as tubulin-interacting agents and DNA-damaging agents. In some embodiments, the cytotoxic agent is a tubulin inhibitor (e.g., a tubulysin). In certain embodiments, the tubulin inhibitor inhibits tubulin polymerization. In some embodiments, the cytotoxic payload is a topoisomerase I inhibitor. In some embodiments, the cytotoxic agent is a maytansinoid, an auristatin, a hemiasterlin, a vinblastine, a vincristine, a pyrrolobenzodiazepine, a paclitaxel, a docetaxel, a cryptophycin, a tubulysin, or a camptothecin analog. Examples of suitable cytotoxic agents and chemotherapeutic agents that can be conjugated to anti-TAA antibodies in accordance with this aspect of the disclosure also include, e.g., 1-(2chloroethyl)-1,2-dimethanesulfonyl hydrazide, 1,8-dihydroxy-bicyclo[7.3.1]trideca-4,9-diene-2,6-diyne-13-one, 1-dehydrotestosterone, 5-fluorouracil, 6-mercaptopurine, 6-thioguanine, 9-amino camptothecin, actinomycin D, amanitins, aminopterin, anguidine, anthracycline, anthramycin (AMC), bleomycin, busulfan, butyric acid, calicheamicins (e.g., calicheamicin yl), camptothecin, carminomycins, carmustine, cemadotins, cisplatin, colchicin, combretastatins, cyclophosphamide, cytarabine, cytochalasin B, Dxd or derivative thereof, dactinomycin, daunorubicin, decarbazine, diacetoxypentyldoxorubicin, dibromomannitol, dihydroxy anthracin dione, disorazoles, dolastatin (e.g., dolastatin 10), doxorubicin, duocarmycin, echinomycins, eleutherobins, emetine, epothilones, esperamicin, estramustines, ethidium bromide, etoposide, fluorouracils, geldanamycins, gramicidin D, glucocorticoids, irinotecans, kinesin spindle protein (KSP) inhibitors, leptomycins, leurosines, lidocaine, lomustine (CCNU), maytansinoids, mechlorethamine, melphalan, mercatopurines, methopterins, methotrexate, mithramycin, mitomycin, mitoxantrone, N8-acetyl spermidine, podophyllotoxins, procaine, propranolol, pteridines, puromycin, pyrrolobenzodiazepines (PBDs), rhizoxins, streptozotocin, tallysomycins, taxol, tenoposide, tetracaine, thioepa chlorambucil, tomaymycins, topotecans, tubulysin, vinblastine, vincristine, vindesine, vinorelbines, and derivatives of any of the foregoing. According to certain embodiments, the cytotoxic agent that is conjugated to an anti-TAA antibody is a maytansinoid such as DM1 or DM4, a tomaymycin derivative, or a dolastatin derivative. According to certain embodiments, the cytotoxic agent that is conjugated to an anti-TAA antibody is an auristatin such as MMAE, MMAF, or derivatives thereof. In some embodiments, the cytotoxic agent is Dxd or a derivative thereof. In some embodiments, the cytotoxic agent is AZ13599185 (see, e.g., Li et. al., 2016 Cancer Cell 29, 117-129). Other cytotoxic agents known in the art are contemplated within the scope of the present disclosure, including, e.g., protein toxins such ricin, C. difficile toxin, pseudomonas exotoxin, ricin, diphtheria toxin, botulinum toxin, bryodin, saporin, pokeweed toxins (i.e., phytolaccatoxin and phytolaccigenin), and others such as those set forth in Sapra et al., Pharmacol. & Therapeutics, 2013, 138:452-469. In some embodiments, the cytotoxic agent is a tubulysin, a maytansinoid, or a camptothecin, or an analog thereof.

In certain example embodiments, the effector agent includes a detectable label. Suitable detectable labels include, for example, radio-opaque labels, nanoparticles, PET labels, MRI labels, radioactive labels, and the like. Among the radionuclides useful in various embodiments of the present invention are gamma-emitters, positron-emitters, x-ray emitters and fluorescence-emitters are suitable for localization, diagnosis and/or staging, and/or therapy, while beta and alpha-emitters and electron and neutron-capturing agents, such as boron and uranium, also can be used for therapy.

The detectable labels, for example, can be used in conjunction with an external detector and/or an internal detector and provide a means of effectively localizing and/or visualizing prostate cancer cells. Such detection/visualization can be useful in various contexts including, for example, pre-operative and intraoperative settings. Thus, in certain example embodiments provided is a method of intraoperatively detecting cancers in the body of a subject. These methods typically involve administering to the subject a composition including, in a quantity sufficient for detection by a detector (e.g., a gamma detecting probe), a cancer specific antibody molecule labeled with a detectable label (e.g., the antibody molecules described herein labeled with a radioisotope, e.g. ¹⁶¹Tb, ¹²³I, ¹²⁵I, and the like). And, after allowing the immunoconjugate to be taken up by the target tissue, for example, subjecting the subject to a radioimmunodetection technique in the relevant area of the body, e.g., by using a gamma detecting probe.

In certain example embodiments, the effector agent is joined to the antibody molecule via a linker. Linkers are any group or moiety that links, connects, or otherwise bonds the antibody molecules described herein with the effector agent (e.g., a therapeutic moiety, such as a cytotoxic agent). That is, any linker molecule or linker technology known in the art can be used to create or construct an ADC of the present disclosure. Suitable linkers may be found, for example, in Antibody-Drug Conjugates and Immunotoxins; Phillips, G. L., Ed.; Springer Verlag: New York, 2013; Antibody-Drug Conjugates; Ducry, L., Ed.; Humana Press, 2013; Antibody-Drug Conjugates; Wang, J., Shen, W.-C., and Zaro, J. L., Eds.; Springer International Publishing, 2015, the contents of each incorporated herein in their entirety by reference.

In certain example embodiments, suitable linkers for the immunoconjugates described herein are those that are sufficiently stable to exploit the circulating half-life of the antibody. In certain embodiments, the linkers are stable in physiological conditions. In certain embodiments, the linkers are non-cleavable. In certain example embodiments, the linkers are cleavable, for instance, able to release at least the payload portion in the presence of an enzyme or at a particular pH range or value. In some embodiments, the linker includes an enzyme-cleavable moiety. Illustrative enzyme-cleavable moieties include, for example, peptide bonds, ester linkages, hydrazones, and disulfide linkages. In some embodiments, the linker includes a cathepsin-cleavable linker.

In certain example embodiments, the linker includes a labile linker, an acid labile linker, a photolabile linker, a charged linker, a disulfide-containing linker, a peptidase-sensitive linker, a β-glucuronide-linker, a dimethyl linker, a thio-ether linker, a hydrophilic linker, an oligopeptide linker (including cleavable and non-cleavable oligopeptide linkers), a hydrazine linker, a thiourea linker, a self-immolative linker, a succinimidyl trans-4-(maleimidylmethyl)cyclohexane-1-carboxylate (SMCC) linker, a maleimide linker, or the like. Cleavable oligopeptide linkers include protease- or matrix metalloprotease-cleavable linkers. In certain example embodiments, the linker can include combinations of the linker described herein. The skilled person understands that further linkers may be suitable.

In certain example embodiments, an effector agent can be indirectly linked to an antibody molecule described herein by methods known in the art. As an example of such indirect binding, the effector agent can be joined to an antibody molecule as described herein via a biotin-avidin/streptavidin linkage (or other similar affinity-based linkage). For example, an antibody molecule as provided herein can be biotinylated by any means known in the art. Thereafter, an effector molecule that includes avid/streptavidin can be used to join the effector molecule to the antibody molecule via a biotin-avidin/streptavidin linkage. In other example embodiments, the antibody molecule can be linked to an avid or streptavidin moiety, with the effector agent being biotinylated. The biotinylated effector agent can then be joined to the antibody molecule via a biotin-avidin/streptavidin linkage.

In certain example embodiments, the effector agent can be covalently coupled to the antibody molecules provided herein, such as via a SpyTag-Spy Catcher linkage pair. For example, a fusion protein including an antibody molecule as provided herein and a SpyCatcher sequence can be recombinantly produced. Thereafter, an effector agent attached to a SpyTag sequence can be brought into contact with the antibody molecule/SpyCatcher fusion peptide, thereby allowing the SpyTag-SpyCatcher bond to form (and hence joining the effector agent to the antibody molecule). See, e.g., Li et al, J Mol Biol. 2014 Jan. 23; 426(2):309-17 (discussing the SpyTag-SpyCatcher system), which is incorporated herein in its entirety. Useful SpyCatcher proteins are described in Kang et al. 2007, Science 318:1625-1628; Zakeri et al., 2012, Proc. Natl. Acad. Sci. USA 109(12):E690-E697; Keeble, 2019, Proc. Natl. Acad. Sci. USA 116:26523-26533; US 2013/0053544 A1; and US 2020/0131233 A1, the contents of which are hereby incorporated by reference in their entireties. In certain embodiments, the SpyCatcher protein is according to Genbank locus JQ478411.1 or Zakeri et al., 2012, Proc. Natl. Acad. Sci. USA 109(12):E690-E697.

### Compositions

In addition to the above embodiments, provided herein are pharmaceutical and veterinary compositions that include, as active ingredients, the antibody molecules and immunoconjugates described herein. That is, the compositions can be prepared from any of the antibody molecules and/or immunoconjugates thereof described herein. In certain example embodiments, the compositions can also include more than one active ingredient as necessary for the particular indication being treated, optionally those with complementary activities that do not adversely affect each other. Additionally or alternatively, the compositions including an antibody molecule as described herein can include an agent that enhances its function, such as, for example, a cytotoxic agent, cytokine, chemotherapeutic agent, or growth-inhibitory agent. Such molecules are suitably present in combination in amounts that are effective for the purpose intended.

As those skilled in the arts will appreciated, such compositions typically include, for example, suitable physiologically compatible and pharmaceutical acceptable excipients such as buffers and other simple excipients, as well as—in certain example embodiments—any pharmaceutically acceptable carrier. The compositions may include additional active ingredients as well, in particular anti-tumor chemotherapeutic agents. The antibody molecules and immunoconjugates described herein can also be used in diagnostic compositions.

In certain example embodiments, the compositions include a carrier for the antibody molecules or immunoconjugates thereof, and desirably a pharmaceutically acceptable carrier as described herein. For example, the pharmaceutically acceptable carrier can be any suitable pharmaceutically acceptable carrier, such as one or more compatible solid or liquid fillers, diluents, other pharmaceutical acceptable excipients, or encapsulating substances which are suitable for administration into a human or veterinary patient (e.g., a physiologically acceptable carrier or a pharmacologically acceptable carrier). The term “carrier” denotes an organic or inorganic ingredient, natural or synthetic, with which the active ingredient is combined to facilitate the use of the active ingredient, e.g., the administration of the active ingredient to a subject. The pharmaceutically acceptable carrier can be co-mingled with one or more of the active components, e.g., a hybrid molecule, and with each other, when more than one pharmaceutically acceptable carrier is present in the composition, in a manner so as not to substantially impair the desired pharmaceutical efficacy. Pharmaceutically acceptable materials typically are capable of administration to a subject, e.g., a patient, without the production of significant undesirable physiological effects such as nausea, dizziness, rash, or gastric upset. It is desirable for a composition comprising a pharmaceutically acceptable carrier not to be immunogenic when administered to a human patient for therapeutic purposes.

The compositions provided herein can additionally contain suitable buffering agents, including, for example, acetic acid in a salt, citric acid in a salt, boric acid in a salt, and phosphoric acid in a salt. The compositions can also optionally contain suitable preservatives, such as benzalkonium chloride, chlorobutanol, parabens, and thimerosal. Pharmaceutical compositions of the invention can be presented in unit dosage form and can be prepared by any suitable method, many of which are well known in the art of pharmacy. Such methods include, for example, the step of bringing the antibody molecules or immunoconjugates thereof into association with a carrier that constitutes one or more accessory ingredients. In general, the composition is prepared by uniformly and intimately bringing the active agent into association with a liquid carrier, a finely divided solid carrier, or both, and then, if necessary, shaping the product.

As those skilled in the art will appreciate, a pharmaceutical or veterinary composition is formulated to be compatible with its intended route of administration. Examples of routes of administration include parenteral, e.g., intravenous, intradermal, subcutaneous, oral (e.g. inhalation), transdermal (i.e., topical), transmucosal, and rectal administration. A composition suitable for parenteral administration, for example, conveniently includes a sterile aqueous preparation of the antibody molecules or immunoconjugates thereof described herein, which preferably is isotonic with the blood of the recipient. This aqueous preparation can be formulated according to known methods using suitable dispersing or wetting agents and suspending agents. The sterile injectable preparation also can be a sterile injectable solution or suspension in a non-toxic parenterally-acceptable diluent or solvent, for example, as a solution in 1,3-butane diol. Among the acceptable vehicles and solvents that can be employed are water, Ringer's solution, and isotonic sodium chloride solution. In addition, sterile, fixed oils are conventionally employed as a solvent or suspending medium. For this purpose, any bland fixed oil can be employed, such as synthetic mono- or di-glycerides. In addition, fatty acids such as oleic acid can be used in the preparation of injectables. Carrier formulations suitable for oral, subcutaneous, intravenous, intramuscular, etc. administrations can be found in Remington's Pharmaceutical Sciences, by E. W. Martin, Mack Publishing Co., Easton, PA, 19th Edition (1995).

As those skilled in the art will also appreciate, preparation of the pharmaceutical and veterinarian compositions described herein, and their various routes of administration, can be carried out in accordance with methods well known in the art. See, e.g., Remington: The Science and Practice of Pharmacy, Mack Publishing Co., 20th ed., 2000; and Sustained and Controlled Release Drug Delivery Systems, J. R. Robinson, ed., Marcel Dekker, Inc., New York, 1978, each of which are incorporated herein in their entirety. The delivery systems useful in the context of the invention include time-released, delayed release, and sustained release delivery systems such that the delivery of the inventive composition occurs prior to, and with sufficient time to cause, sensitization of the site to be treated. The composition provided herein can be used in conjunction with other therapeutic agents or therapies. Such systems can avoid repeated administrations of the inventive composition, thereby increasing convenience to the subject and the physician, and may be particularly suitable for certain compositions of the invention.

Many types of release delivery systems are available and known in the art. Suitable release delivery systems include polymer base systems such as poly(lactide-glycolide), copolyoxalates, polycaprolactones, polyesteramides, polyorthoesters, polyhydroxybutyric acid, and poly anhydrides, and microcapsules. Delivery systems also include non-polymer systems that are lipids including sterols such as cholesterol, cholesterol esters, and fatty acids or neutral fats such as mono-di- and triglycerides; hydrogel release systems; sylastic systems; peptide-based systems; wax coatings; compressed tablets using conventional binders and excipients; partially fused implants; and the like.

In certain example embodiments the antibody molecules and/or immunoconjugates thereof can be administered in the “native” form or, if desired, in the form of salts, esters, amides, prodrugs, derivatives, and the like, provided the salt, ester, amide, prodrug or derivative is suitable pharmacologically, i.e., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience, and as described above.

### Methods of Use

The antibody molecules and immunoconjugates thereof described herein have a variety of therapeutic uses. In certain example embodiments, provided is a method of treating or preventing a disease or condition in a subject associated with, or characterized by, the expression of Anaplastic Lymphoma Kinase (ALK). The method includes, for example, administering to the subject a therapeutically effective amount of one or more of the antibody molecules, immunoconjugates, or compositions thereof described herein, thereby treating the subject.

In certain example embodiments, provided is a method for treating a cancer, reducing tumor growth, and/or causing tumor regression in a subject. In certain example embodiments, the cancer is an ALK-expressing cancer, i.e., the cancer may have increased ALK expression, or be known to be associated with increased ALK expression, such as relative to a control. The method includes, for example, administering to a subject in need thereof therapeutically effective amount of one or more of the antibody molecules, immunoconjugates, or compositions thereof described herein, thereby treating the subject. In certain example embodiments, such administration can be used to treat or prevent a cancer recurrence.

In certain example embodiments, provided is a method for targeting a cancer cell, such as to kill or reduce the proliferation of the cancer cell. To target the cancer cell, for example, a therapeutically effective amount of one or more of the antibody molecules, immunoconjugates, or compositions thereof is administered to a subject, such as a cancer patient. And as with the other embodiments provided herein, the antibody molecule, either alone or as associated with an immunoconjugate or composition thereof, targets the cancer cell by immunospecifically binding to ALK associated with the cancer cell. In certain example embodiments, such administration can be used to treat or prevent a cancer recurrence.

In certain example embodiments, the antibody molecules, immunoconjugates, or compositions thereof described herein are provided for use in treating a disease or condition in a subject associated with, or characterized by, the expression of ALK; treating a cancer, reducing tumor growth, and/or causing tumor regression in a subject; treating or preventing cancer recurrence; or, for use in targeting a cancer cell. That is, such antibody molecules, immunoconjugates, or compositions thereof described herein can be made and provided to an end user, such as a physician or pharmacist, for such intended uses.

In any method or use described herein, the cancer can be any type of cancer where the cancer cells express—or that are known to express—ALK. In certain example embodiments, the cancer may be ovarian cancer (e.g., undifferentiated ovarian cancer, granulosa cell ovarian cancer, endometrioid ovarian cancer, serous ovarian cancer, secondary ovarian cancer (another primary), clear cell ovarian cancer, epithelial tumors, germ cell carcinoma tumors, stromal carcinoma tumors and small cell carcinoma of the ovary), cervical cancer, pancreatic cancer, uterine cancer, esophageal cancer, melanoma cancer, glioblastoma cancer, head and neck cancer, colorectal cancer, bladder cancer, lung cancer, prostate cancer, sarcoma, breast, liver or renal cancer, acute myelogeneous leukemia, or melanoma.

In certain preferred example embodiments, the cancer is a hematopoietic malignancy, such as Acute lymphoblastic leukemia (ALL), Acute lymphoblastic leukemia (ALL), Chronic lymphocytic leukemia (CLL), Chronic myelogenous leukemia (CML), Acute monocytic leukemia (AMoL), or other leukemia. In other preferred example embodiments, the cancer is a myeloma, such as Light Chain Myeloma, Non-secretory Myeloma, Solitary Plasmacytoma, Extramedullary Plasmacytoma, Monoclonal Gammopathy of Undetermined Significance (MGUS), Smoldering Multiple Myeloma (SMM), Immunoglobulin D (IgD) Myeloma, or Immunoglobulin E (IgE) Myeloma. In certain example embodiments, the cancer is a lymphoma, i.e., a Hodgkin lymphoma or a non-Hodgkin lymphoma (e.g., indolent, aggressive, or highly aggressive). In still other preferred example embodiments, the cancer us a non-hematopoietic malignancies. For example, the cancer can be a neuroblastoma, lung cancer (e.g., adenocarcinoma), thyroid cancer, glioblastoma, or rhabdomyosarcoma.

As those skilled in the art will appreciate based on this disclosure, treatment with the antibody molecules, immunoconjugates, or compositions thereof provided herein will result in an improvement in the signs or symptoms of disease. For instance, where the disease being treated is cancer, such therapy may result in an improvement in survival (overall survival and/or progression free survival) and/or may result in an objective clinical response (partial or complete). In certain example embodiments, the treatment may result in decreased tumor size, tumor number, or tumor burden.

In certain preferred embodiments, the antibody molecules provided herein are administered as a naked antibody or antigen binding fragment thereof. That is, the antibody molecules are administered without being conjugated to an effector agent as described herein. In other example embodiments, however, the antibody molecules provided herein are provided as immunoconjugates, wherein the effector agent is an agent described herein useful for treating an identified disease state. For example, if the disease state is cancer, the administered antibody molecules provided herein may be conjugated with a cytotoxic agent as described herein. Preferably, the immunoconjugate and/or antibody molecule to which it is bound is/are internalized by the cell, resulting in increased therapeutic efficacy of the immunoconjugate in killing the cancer cell to which it binds. In a preferred embodiment, the cytotoxic agent targets or interferes with nucleic acid in the cancer cell and/or otherwise disrupts the cell's division cycle.

In certain example embodiments, the antibody molecules, and/or immunoconjugates thereof, are rapidly internalized to the target cells. For example, the antibody molecules, and/or immunoconjugates thereof are substantially internalized over the course of about 15, 20, 25, 30, 35, 40, 45, 50, 55, 60, 70, 80, 90, 100, 110, or 120 minutes. More preferably, the antibody molecules, and/or immunoconjugates thereof are substantially internalized into a target cell, such as a target cell, in 20-40 minutes, such as about 30 minutes. For example, after about 20-40 minutes, and more preferably after about 30 minutes, more than about 70-90% of the antibody molecules, and/or immunoconjugates thereof, are internalized into a target cell. In certain example embodiments, after about 20-40 minutes, and more preferably after about 30 minutes, more than about 80% of the antibody molecules, and/or immunoconjugates thereof, are internalized into a target cell.

In certain example embodiments provided is a method for detecting a cancer, such as by administering to a subject an immunoconjugate, the immunoconjugate including an antibody molecule as described herein and a detectable label (as the effector agent). The label is then detected, such as after a time when the immunoconjugate has had tome to localize to the cancer. Detecting the location of the label then corresponds to detecting the location of a tumor, for example.

In certain example embodiments, the immunoconjugates provided herein can be produced in vivo or in situ, thus temporally spacing administration of an antibody molecule as provided herein with targeting of the effector agent provided herein to the antibody molecule. That is, an antibody molecule provided herein can be first administered to a subject, so that the antibody molecule is “pretargeted” to ALK expressing cells. Thereafter, the effector agent is targeted to the antibody. See, e.g., Patra et al., New insights into the pretargeting approach to image and treat tumours, Chem. Soc. Rev., 2016, 45, 6415, which is incorporated herein in its entirety. For example, a bi-specific antibody molecule can be engineered in which one specificity binds ALK while a second specificity binds the effector agent, as described herein, with the antibody molecule being first administered to a subject and the effector agent being subsequently administered to the subject. In such example embodiments, the antibody molecule-effector agent forms the immunoconjugate in vivo or in situ, such as at the site of an ALK-expressing cell.

As those skilled in the art will appreciate, any molecule or moiety capable of linking an anybody molecule as provided herein to an effector agent in vivo or in situ can be used in such pretargeting embodiments. In certain example embodiments, pretargeting of the antibody molecules provided herein can be accomplished via the use of a biotin or avidin/streptavidin molecule or moiety. For example, the antibody molecule can be engineered to include a biotin or avidin/streptavidin moiety. The antibody can then be administered to a subject. Thereafter, an effector agent including the reciprocal biotin or avidin/streptavidin binding moiety can be administered to the subject, thus allowing the effector agent to bind to the antibody molecule via an avidin/streptavidin-biotin linkage. Such targeting of the effector agent to the pretargeted antibody molecule thus targets the effector agent to the site on the ALK-expressing cell. Additionally or alternatively, and again as an illustrative example of useful pretargeting systems, the SpyTag/SpyCatcher system can be used in such pretargeting embodiments.

As those skilled in the art will also appreciate, maximum antibody molecule localization to a target cell takes 12-60 hours, and more typically 24-48 hours. By contrast, a labeled biotin or avidin/streptavidin molecule can localize to its binding partner rapidly within a much shorter timeframe, such as within minutes to a few hours (e.g., 15, 20, 25, 30, 35, 40, 45, 50, 55, 60, 70, 80, 90, 100, 110, or 120 minutes). As such, when an effector agent might have toxic side effects (or when decreased exposure time to the effector agent is desired), the pretargeting approach using the antibody molecules/effector agents described herein can reduce toxic exposure time of the effector agent. Similarly, for diagnostic imaging purposes, pretargeting can improve signal to noise.

For example, when a radiolabeled effector agent is used for cancer imaging, the 12-60 hours, and more typically 24-48 hours, of antibody molecule binding time can be used to pretarget an antibody molecule to a ALK-expressing tumor site, without the need for attaching a radiolabel to the antibody molecule (and exposing the subject to the radiolabel for the 12-60 hours, and more typically 24-48 hours). Instead, with the pretargeting approach, a radiolabeled effector agent can be administered some time after the administration of the antibody molecule, thereby decreasing the exposure time of a subject to the effector agent (and any of its toxic effects). For example, with the pretargeting approach, an antibody molecule provided herein including a pair binding moiety (e.g., biotin) can be administer to a subject. Thereafter, an effector agent including the binding partner (e.g., avidin) can be administered to the subject, such as at about 5, 10, 15, 20, 25, 30, 35, 40, 45, 50, 55, 60, 65, or 70 hours after the administration of the antibody molecule including the binding moiety, thus targeting the effector agent to the pretargeted antibody molecule (via, e.g., a biotin-avidin linkage). More preferably, the effector agent is administered at about 20, 25, 30, 35, 40, 45, or 50 hours after the administration of the antibody molecule.

In certain example embodiments, an antibody molecule as provided herein can be expressed in situ or in vivo or in situ, as described herein, allowing the expressed antibody molecule to then target ALK-expressing cells. In such embodiments, the antibody molecule can be engineered as a fusion protein with a binding moiety (e.g., avidin). Thereafter, the expressed antibody molecules can be targeted with an effector agent that includes a moiety with high affinity to the binding moiety (e.g., biotin). In such embodiments, an administered effector agent can target and bind to the antibody molecule, thereby targeting the effector agent to the ALK-expressing cells via the antibody molecule.

### EXAMPLES

The following examples further illustrate the invention but should not be construed as in any way limiting its scope. In light of the present disclosure and the general level of skill in the art, those of skill will appreciate that the following Examples are intended to be exemplary only and that numerous changes, modifications, and alterations can be employed without departing from the scope of the presently disclosed subject matter.

As used herein, the following abbreviations apply: eq (equivalents); M (Molar); μM (micromolar); N (Normal); mol (moles); mmol (millimoles); pmol (micromoles); nmol (nanomoles); g (grams); mg (milligrams); kg (kilograms); μg (micrograms); L (liters); ml (milliliters); μl (microliters); cm (centimeters); mm (millimeters); μm (micrometers); nm (nanometers); ° C. (degrees Centigrade); h (hours); min (minutes); sec (seconds); msec (milliseconds).

### Example 1—Identification & Generation of Anti-ALK Antibodies

Human peripheral blood mononuclear cells (PBMCs) from anonymized donors from the Stanford Blood Center, obtained under informed consent, were screened for binding to the extracellular domain of ALK. The cells were subjected to the CellSpot™ assay to determine their ability to bind this antigen with specificity compared to other antigens. The CellSpot™ assay is described in U.S. Pat. Nos. 7,413,868 and 7,939,344. After isolating the B cells from whole blood, they were stimulated with cytokines and mitogens to initiate a brief period of proliferation, differentiation, and antibody secretion (lasting ˜5 days) and plated for subjection to the assays. The encoding nucleic acids for the variable regions of positive antibodies were extracted and used to produce the antibodies recombinantly by cloning the DNA in expression vectors that contain a signal peptide as well as fusion of the DNA encoding the variable region with DNA cloned independently that codes for the constant region of the antibody.

### Example 2—Cloning & Characterization of Anti-ALK Antibodies

The ALK antibodies of Example 1 were cloned following a survey of 12 blood donors for binding to the extracellular domain of ALK. Anti-ALK antibodies were detected in all donors albeit at different but low frequencies. Table 1 shows the frequencies for each donor tested. BSA was used as a counterscreen to eliminate polyreactive antibodies. Five mAbs were cloned from 4 different donors.

TABLE 1
Donor Frequencies

| | # CellSpots/100K |
|---|---|
| Donor | Memory Cells |
| SBC207 | 1.1 |
| SBC210 | 1.0 |
| SBC222 | 1.6 |
| SBC223 | 5.3 |
| SBC230 | 1.3 |
| SBC236 | 0.9 |
| SBC240 | 2.6 |
| SBC241 | 0.5 |
| SBC242 | 0.7 |
| SBC246 | 2.0 |
| SBC248 | 1.5 |

TABLE 2
Donors for Anti-ALK antibodies.

| | Donor |
|---|---|
| TRLmAb | # |
| 10001 | 248 |
| 10005 | 236 |
| 10006 | 236 |
| 10014 | 210 |
| 10051 | 223 |

Purified mAbs were tested in adsorption ELISA using ALK Extracellullar Domain (ECD), generated by Trellis in mammalian cells. Serial dilutions allowed calculating an estimate of the binding affinities (values listed in Table 3 are expressed as nM). TRL10001 is of particular interest based on its sub-nM affinity to the target. A diverse set of germline variable regions was found in this group of mAbs as seen in Table 4.

TABLE 3
Affinities for Anti-ALK antibodies.

| | Affinity |
|---|---|
| TRLmAb | (nM) |
| 10001 | 0.2 |
| 10005 | 1 |
| 10006 | 62 |
| 10014 | 75 |
| 10051 | 1 |

TABLE 4
Germlines for Anti-ALK antibodies.

| | VH | VL |
|---|---|---|
| TRLmAb | germline | germline |
| 10001 | IGHV5- | IGKV1- |
| | 51*01 | 39*01 |
| 10005 | IGHV3- | IGKV3- |
| | 11*01 | 20*01 |
| 10006 | IGHV3- | IGKV3- |
| | 48*01 | 11*01 |
| 10014 | IGHV3- | IGKV2- |
| | 11*01 | 28*01 |
| 10051 | IGHV3- | IGKV2- |
| | 23*01 | 24*01 |

### Example 3—Assessment of Anti-ALK TRL10001 Binding & Internalization

ALK is appropriate for targeted antibody therapy since it is only expressed in the nervous system during embryogenesis and not in normal tissues (Iwahara, 1997), lowering the chances of toxicity. In contrast, multiple malignancies are known to harbor ALK alterations. Dysregulated ALK expression has been identified in nearly 20 different malignancies. ALK activation occurs through three different mechanisms: 1) formation of fusion protein with Nucleophosphin (Morris, 1994) or Echinoderm Microtubule-associated protein-Like 4, EML4, (Li, 2011); 2) ALK over-expression (Schulte, 2011); and 3) single point mutation in the kinase domain leading to kinase activation (Lovisa, 2015 and Tartan, 2008). Only the first two present ALK on the cell surface and can be targeted with an anti-ALK antibody. One mechanism for resistance to the approved kinase inhibitor drugs is increasing ALK surface display which could benefit for antibody targeted therapy (Carpenter, 2012). Indeed, combination of small molecule receptor tyrosine kinase inhibitors with mAbs to HER2 or EGFR enhances the tumor growth inhibition induced by any of the agents separately (Scaltriti, 2009 and Regales, 2009, respectively). Important cancer types with surface expressed ALK include: non-small-cell lung cancer, NSCLC, Guerin, 2015 and Jiang, 2016), basal cell carcinoma (Ning, 2013), neuroblastoma (Carpenter, 2012 and Osajima-Hakomori, 2005), glioblastoma (Powers, 2002), Ewing's sarcoma (Fleuren, 2013), ovarian cancer (Ren, 2012), inflammatory breast cancer (Robertson, 2013), anaplastic thyroid carcinoma (Murugna, 2011), melanoma (Wiesner, 2014) and, rhabdomyosarcoma (van Gaal, 2012).

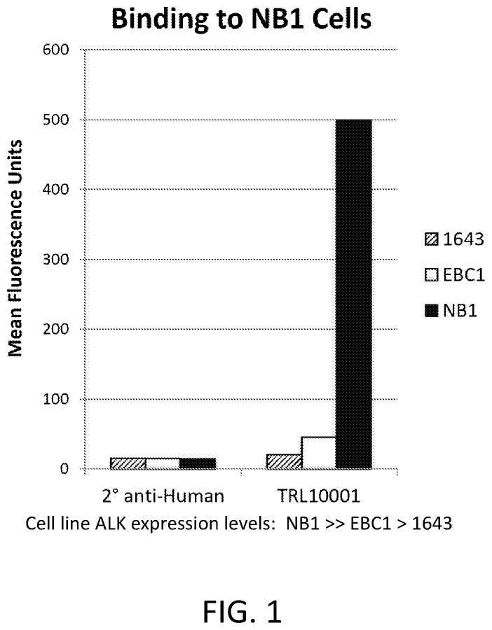

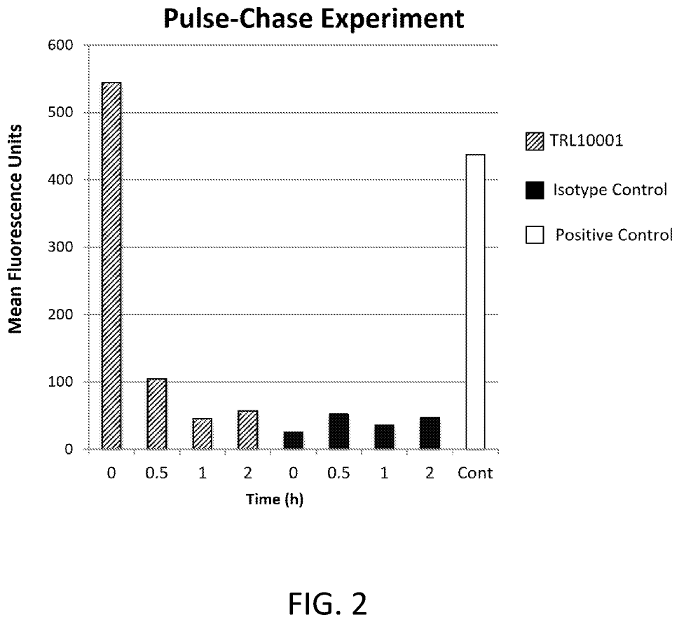

The high affinity human anti-ALK antibody TRL10001 was tested for binding to ALK on the surface of several neuroblastoma cell lines that expressed different levels of ALK with NB1 cells having the highest expression. As expected, staining of this cell line was the highest with TRL10001. In addition, TRL10001 was tested for internalization on NB1 cells (requisite for ADC design). More than 80% of TRL10001 was internalized in 30 minutes of incubation at 37 C. Results are show in FIG. 1 and FIG. 2.

### Example 4—Sequencing of Anti-ALK Antibodies

For each of the antibodies of Table 3 (above), cDNA and associated amino acid sequences were determined by as follows. After identifying a human B-cell secreting a mAb meeting the selection criteria, the encoding mRNAs for heavy and light chains were amplified by single cell RT-PCR from sibling cells and subcloned into the previously described pTT5 vector as an IgG1. The recombinant plasmid was extracted, identified by restriction enzyme digestion, DNA sequenced, and amino acid sequence determined.

Further, for TRL10001, the complementary determining regions (CDRs) were determined. More particularly, IgBLAST program, available from the National Center for Biotechnology Information, was used to determine the IgG V domain using either the ImMunoGeneTics information System® (IMGT) (Lefranc et al 2003) or Kabat system (Kabat et al, 1991). IgBLAST uses the heavy and light chain nucleotide sequence to searched with BLAST (Basic Local Algorithm Search Tool) against the IMGT or NCBI germline V gene database (the sequences in such databases have been pre-annotated for the framework regions (FR)/complementarity determining regions (CDR) boundaries). The top database sequence hit was used to map the pre-annotated IMGT and KABAT FR/CDR boundary information to the query sequence. The summary IgBLAST information includes the identifiers of the best matched V, D and J gene, the relationship between the coding frames of the V and J genes, the details of the V-(D)-J junctions and the match statistics for various FR/CDR (Ye et al, 2013). The results of the IMGT and Kabat method CDR analysis is provided in Table 5 (for TRL10001 heavy chain) and Table 6 (for TRL10001 light chain).

TABLE 5
Alignment Summary Between Query and Top Germline V Gene Hit (IGHV5-
51*01) for TRL10001 heavy chain.

| | | | NT | | | Mis- | | Identity | | |
|---|---|---|---|---|---|---|---|---|---|---|
| Antibody | Method | CDR | Position | Length | Matches | matches | Gaps | (%) | NT Sequence | AA Sequence |
| TRL10001 | IMGT | CDR-H1 | 76 to 99 | 24 | 22 | 2 | 0 | 91.7 | GGATTCAGCTTTAC | GFSFINYW |
| Heavy | | | | | | | | | CAACTACTGG | (SEQ ID NO: 2) |
| Chain | | | | | | | | | (SEQ ID NO: 16) | |
| | | CDR-H2 | 151 to 174 | 24 | 22 | 2 | 0 | 91.7 | ATCTTTCTTGGTGA | IFLGDSDT |
| | | | | | | | | | CTCTGATACC | (SEQ ID NO: 3) |
| | | | | | | | | | (SEQ ID NO: 17) | |
| | | CDR-H3 | 289 to 336 | 48 | | | | | GCGCGACCCAAAG | ARPKGGGWNDF |
| | | | | | | | | | GAGGCGGCTGGAA | SGFEI |
| | | | | | | | | | CGACTTCTCTGGTT | (SEQ ID NO: 4) |
| | | | 289 to 295 | 7 | 6 | 1 | 0 | 85.7 | TTGAAATC | |
| | | | (germline) | | | | | | (SEQ ID NO: 18) | |
| | KABAT | CDR-H1 | 91 to 105 | 15 | 14 | 1 | 0 | 93.3 | AACTACTGGATCGG | NYWIG |
| | | | | | | | | | C | (SEQ ID NO: 5) |
| | | | | | | | | | (SEQ ID NO: 19) | |
| | | CDR-H2 | 148 to 198 | 51 | 47 | 4 | 0 | 92.2 | ATGATCTTTCTTGG | MIFLGDSDTRYSP |
| | | | | | | | | | TGACTCTGATACCA | SFRG |
| | | | | | | | | | GATACAGCCCGTCC | (SEQ ID NO: 6) |
| | | | | | | | | | TTCCGAGGC | |
| | | | | | | | | | (SEQ ID NO: 20) | |
| | | CDR-H3 | 295 to 336 | 42 | 1 | 0 | 0 | 100 | CCCAAAGGAGGCG | PKGGGWNDFSGF |
| | | | 295 | 1 | | | | | GCTGGAACGACTTC | EI |
| | | | (V gene | | | | | | TCTGGTTTTGAAAT | (SEQ ID NO: 7) |
| | | | only) | | | | | | C | |
| | | | | | | | | | (SEQ ID NO: 21) | |

TABLE 6
Alignment Summary Between Query and Top Germline V Gene Hit (IGHV5-
51*01) for TRL10001 light chain.

| | | | | | | Mis- | | Identity | | |
|---|---|---|---|---|---|---|---|---|---|---|
| Antibody | Method | CDR | NT Position | Length | Matches | matches | Gaps | (%) | NT Sequence | AA Sequence |
| TRL10001 | IMGT | CDR- | 79 to 96 | 18 | 13 | 5 | 0 | 72.2 | CAGAGCATTACAAA | QSITKF |
| Light | | L1 | | | | | | | GTTT | (SEQ ID) NO: 9) |
| Chain | | | | | | | | | (SEQ ID NO: 23) | |
| | | CDR- | 148 to 156 | 9 | 9 | 0 | 0 | 100 | GCTGCATCC (SEQ | AAS |
| | | L2 | | | | | | | ID NO: 24) | (SEQ ID NO: 10) |
| | | CDR- | 265 to 291 | 27 | | | | | CAACAGAGTTTCAG | QQSFSTPRP |
| | | L3 | 265 to 286 | 22 | 20 | 2 | 0 | 90.9 | CACCCCTCGGCCG | (SEQ ID NO: 11) |
| | | | (germline) | | | | | | (SEQ ID NO: 25) | |
| | KABAT | CDR- | 70 to 102 | 33 | 27 | 6 | 0 | 81.8 | CGGGCGAGTCAGA | RASQSITKFLN |
| | | L1 | | | | | | | GCATTACAAAGTTT | (SEQ ID NO: 12) |
| | | | | | | | | | TTAAAT | |
| | | | | | | | | | (SEQ ID NO: 26) | |
| | | CDR- | 148 to 168 | 21 | 21 | 0 | 0 | 100 | GCTGCATCCAGTTT | AASSLQS |
| | | L2 | | | | | | | GCAAAGT | (SEQ ID NO: 13) |
| | | | | | | | | | (SEQ ID NO: 27) | |
| | | CDR- | 265 to 291 | 27 | | | | | CAACAGAGTTTCAG | QQSFSTPRP |
| | | L3 | 265 to 286 | 22 | 20 | 2 | 0 | 90.9 | CACCCCTCGGCCG | (SEQ ID NO:14) |
| | | | (V gene | | | | | | (SEQ ID NO: 28) | |
| | | | only) | | | | | | | |

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

### REFERENCES

Each of the following references, along with any other references identified herein, are hereby incorporated herein in their entirety.
1. Carpenter E L, Haglund E A, Mace E M, Deng D, Martinez D, Wood A C, Chow A K, Weiser D A, Belcastro L T, Winter C, Bresler S C, Asgharzadeh S, Seeger R C, Zhao H, Guo R, Christensen J G, Orange J S, Pawel B R, Lemmon M A and Mosse Y P. Antibody targeting of anaplastic lymphoma kinase induces cytotoxicity of human neuroblastoma. Oncogene. 2012; 31(46): 4859-4867.
2. Carpenter E L, Mosse Y P. Targeting ALK in neuroblastoma—preclinical and clinical advancements. Nat Rev Clin Oncol. 2012; 9(7):391-9.
3. Fleuren E D, Roeffen M H, Leenders W P, Flucke U E, Vlenterie M, Schreuder H W, Boerman O C, van der Graaf W T and Versleijen-Jonkers Y M. Expression and clinical relevance of MET and ALK in Ewing sarcomas. Int J Cancer. 2013; 133(2):427-36.
4. Guerin A, Sasane M, Zhang J, Macalalad A R, Galebach P, Jarvis J, Kageleiry A, Culver K, Wu E Q and Wakelee H. ALK rearrangement testing and treatment patterns for patients with ALK-positive non-small cell lung cancer. Cancer Epidemiol. 2015; 39(3):307-12.
5. Iwahara T, Fujimoto J, Wen D, Cupples R, Bucay N, Arakawa T, Mori S, Ratzkin B and Yamamoto T. Molecular characterization of ALK, a receptor tyrosine kinase expressed specifically in the nervous system. Oncogene. 1997; 14:439-449.
6. Jiang L, Yang H, He P, Liang W, Zhang J, Li J, Liu Y and He J. Improving Selection Criteria for ALK Inhibitor Therapy in Non-Small Cell Lung Cancer: A Pooled-Data Analysis on Diagnostic Operating Characteristics of Immunohistochemistry. Am J Surg Pathol. 2016; 40(5):697-703.
7. Kwak E L, Bang Y J, Camidge D R, Shaw A T, Solomon B, Maki R G, Ou S H, Dezube B J, Janne P A, Costa D B, Varella-Garcia M, Kim W H, Lynch T J, Fidias P, Stubbs H, Engelman J A, Sequist L V, Tan W, Gandhi L, Mino-Kenudson M, Wei G C, Shreeve S M, Ratain M J, Settleman J, Christensen J G, Haber D A, Wilner K, Salgia R, Shapiro G I, and Iafrate A J. Anaplastic lymphoma kinase inhibition in non-small-cell lung cancer. N Engl J Med. 2010; 363(18):1693-703.
8. Li Y, Ye X, Liu J, Zha J and Pei L. Evaluation of EML4-ALK fusion proteins in non-small cell lung cancer using small molecule inhibitors. Neoplasia. 2011; 13(1):1-11.
9. Lovisa F, Cozza G, Cristiani A, Cuzzolin A, Albiero A, Mussolin L, Pillon M, Moro S, Basso G, Rosolen A and Bonvini P. ALK kinase domain mutations in primary anaplastic large cell lymphoma: consequences on NPM-ALK activity and sensitivity to tyrosine kinase inhibitors. PLoS One. 2015; 10(4):e0121378.
10. Morris S W, Kirstein M N, Valentine M B, Dittmer K G, Shapiro D N, Saltman D L and Look A T. Fusion of a kinase gene, ALK, to a nucleolar protein gene, NPM, in non-Hodgkin's lymphoma. Science. 1994; 263(5151):1281-4.
11. Marsilje T H, Pei W, Chen B, Lu W, Uno T, Jin Y, Jiang T, Kim S, Li N, Warmuth M, Sarkisova Y, Sun F, Steffy A, Pferdekamper A C, Li A G, Joseph S B, Kim Y, Liu B, Tuntland T, Cui X, Gray N S, Steensma R, Wan Y, Jiang J, Chopiuk G, Li J, Gordon W P, Richmond W, Johnson K, Chang J, Groessl T, He Y Q, Phimister A, Aycinena A, Lee C C, Bursulaya B,
12. Karanewsky D S, Seidel H M, Harris J L, and Michellys P Y. Synthesis, structure-activity relationships, and in vivo efficacy of the novel potent and selective anaplastic lymphoma kinase (ALK) inhibitor 5-chloro-N2-(2-isopropoxy-5-methyl-4-(piperidin-4-yl)phenyl)-N4-(2-(isopropylsulfonyl)phenyl)pyrimidine-2,4-diamine (LDK378) currently in phase 1 and phase 2 clinical trials. J Med Chem. 2013; 56(14):5675-90.
13. Murugan A K and Xing M. Anaplastic thyroid cancers harbor novel oncogenic mutations of the ALK gene. Cancer Res. 2011; 71(13):4403-11.
14. Ning H, Mitsui H, Wang C Q, Suárez-Fariñas M, Gonzalez J, Shah K R, Chen J, Coats I, Felsen D, Carucci J A and Krueger J G. Identification of anaplastic lymphoma kinase as a potential therapeutic target in Basal Cell Carcinoma. Oncotarget. 2013; 4(12):2237-48.
15. Osajima-Hakomori Y, Miyake I, Ohira M, Nakagawara A, Nakagawa A and Sakai R. Biological role of anaplastic lymphoma kinase in neuroblastoma. Am J Pathol. 2006; 167(1):213-22.
16. Powers C, Aigner A, Stoica G E, McDonnell K and Wellstein A. Pleiotrophin signaling through anaplastic lymphoma kinase is rate-limiting for glioblastoma growth. J Biol Chem. 2002; 277(16):14153-8.
17. Regales L, Gong Y, Shen R, de Stanchina E, Vivanco I, Goel A, Koutcher J A, Spassova M, Ouerfelli O, Mellinghoff I K, Zakowski M F, Politi K A and Pao W. Dual targeting of EGFR can overcome a major drug resistance mutation in mouse models of EGFR mutant lung cancer. J Clin Invest. 2009; 119(10):3000-10.
18. Ren H, Tan Z P, Zhu X, Crosby K, Haack H, Ren J M, Beausoleil S, Moritz A, Innocenti G, Rush J, Zhang Y, Zhou X M, Gu T L, Yang Y F and Comb M J. Identification of anaplastic lymphoma kinase as a potential therapeutic target in ovarian cancer. Cancer Res. 2012; 72(13):3312-23.
19. Robertson F M, Petricoin E F, Van Laere S J, Bertucci F, Chu K, Fernandez S V, Mu Z, Alpaugh K, Pei J, Circo R, Wulfkuhle J, Ye Z, Boley K M, Liu H, Moraes R, Zhang X, Demaria R, Barsky S H, Sun and Cristofanilli M. Presence of anaplastic lymphoma kinase in inflammatory breast cancer. Springerplus. 2013 2:497
20. Sakamoto H, Tsukaguchi T, Hiroshima S, Kodama T, Kobayashi T, Fukami T A, Oikawa N, Tsukuda T, Ishii N, Aoki Y. CH5424802, a selective ALK inhibitor capable of blocking the resistant gatekeeper mutant. Cancer Cell. 2011 19(5):679-90.
21. Scaltriti M, Verma C, Guzman M, Jimenez J, Parra J L, Pedersen K, Smith D J, Landolfi S, Ramon y Cajal S, Arribas J and Baselga J. Lapatinib, a HER2 tyrosine kinase inhibitor, induces stabilization and accumulation of HER2 and potentiates trastuzumab-dependent cell cytotoxicity. Oncogene. 2009 28(6):803-14.
22. Schulte J H, Bachmann H S, Brockmeyer B, Depreter K, Oberthur A, Ackermann S, Kahlert Y, Pajtler K, Theissen J, Westermann F, Vandesompele J, Speleman F, Berthold F, Eggert A, Brors B, Hero B, Schramm A and Fischer M. High ALK receptor tyrosine kinase expression supersedes ALK mutation as a determining factor of an unfavorable phenotype in primary neuroblastoma. Clin Cancer Res. 2011; 17(15):5082-92.
23. Tartan C J, Gunby R H, Coluccia A M, Sottocomola R, Cimbro B, Scapozza L, Donella-Deana A, Pinna L A and Gambacorti-Passerini C. Characterization of some molecular mechanisms governing autoactivation of the catalytic domain of the anaplastic lymphoma kinase. J Biol Chem. 2008; 283(7):3743-50.
24. van Gaal J C, Flucke U E, Roeffen M H, de Bont E S, Sleijfer S, Mavinkurve-Groothuis A M, Suurmeijer A J, van der Graaf W T and Versleijen-Jonkers Y M. Anaplastic lymphoma kinase aberrations in rhabdomyosarcoma: clinical and prognostic implications. J Clin Oncol. 2012; 30(3):308-15.
25. Wang Y W, Tu P H, Lin K T, Lin S C, Ko J Y and Jou Y S. Identification of oncogenic point mutations and hyperphosphorylation of anaplastic lymphoma kinase in lung cancer. Neoplasia. 2011; 13(8):704-15.
26. Wiesner T, He J, Yelensky R, Esteve-Puig R, Botton T, Yeh I, Lipson D, Otto G, Brennan K, Murali R, Garrido M, Miller V A, Ross J S, Berger M F, Sparatta A, Palmedo G, Cerroni L, Busam K J, Kutzner H, Cronin M T, Stephens P J and Bastian B C. Kinase fusions are frequent in Spitz tumours and spitzoid melanomas. Nat Commun. 2014; 5:3116.
27. Lefranc M P, Pommie C, Ruiz M, Giudicelli V, Foulquier E, Truong L, Thouvenin-Contet V, Lefranc G. IMGT unique numbering for immunoglobulin and T cell receptor variable domains and Ig superfamily V-like domains. Dev Comp Immunol. 2003 January; 27(1):55-77.
28. Kabat E. A., Wu, T. T., Perry, H., Gottesman, K. and Foeller, C. Sequences of Proteins of Immunological Interest, Fifth Edition. 1991 NIH Publication No. 91-3242.
29. Ye J, Ma N, Madden T L, Ostell J M. IgBLAST: an immunoglobulin variable domain sequence analysis tool. Nucleic Acids Res. 2013 July; 41 (Web Server issue):W34-40.

### Sequence Listing Free Text (TRL 10001)

SEQ ID NO: 1 (TRL10001 VH Region)

| | |
|---|---|
| QVQLVQSGAE VKKPGESLKI SCLGSGFSFT NYWIGWVRQM PGKGLESMGM | 50 |
| IFLGDSDTRY SPSFRGQVTI SADKSISTAY LQWSSLRASD TAMYYCARPK | 100 |
| GGGWNDFSGF EIWGQGTMVT VSS | 123 |

SEQ ID NO: 2 (TRL10001 VH CDR-H1--IMGT)
GFSFTNYW
SEQ ID NO: 3 (TRL10001 VH CDR-H2--IMGT)
IFLGDSDT
SEQ ID NO: 4 (TRL10001 VH CDR-H3--IMGT)
ARPKGGGWNDFSGFEI
SEQ ID NO: 5 (TRL10001 VH CDR1-H1--KABAT)
NYWIG
SEQ ID NO: 6 (TRL10001 VH CDR-H2--KABAT)
MIFLGDSDTRYSPSFRG
SEQ ID NO: 7 (TRL10001 VH CDR-H3--KABAT)
PKGGGWNDFSGFEI
SEQ ID NO: 8 (TRL10001 VL Kappa)

| | |
|---|---|
| DITLTQSPTS LSASVGDRVT ITCRASQSIT KFLNWYQQKP GKVPKLLINA | 50 |
| ASSLQSGVPS RFSGSGSGTD YTLTISNLQP EDFATYYCQQ SFSTPRPFGQ | 100 |
| GTRVEIKR | 108 |

SEQ ID NO: 9 (TRL10001 VL CDR-L1--IMGT)
QSITKF
SEQ ID NO: 10 (TRL10001 VL CDR-L2--IMGT)
AAS
SEQ ID NO: 11 (TRL10001 VL CDR-L3--IMGT)
QQSFSTPRP
SEQ ID NO: 12 (TRL10001 VL CDR-L1--KABAT)
RASQSITKFLN
SEQ ID NO: 13 (TRL10001 VL CDR-L2--KABAT)
AASSLQS
SEQ ID NO: 14 (TRL10001 VL CDR-L3--KABAT)
QQSFSTPRP
SEQ ID NO: 15 (TRL10001 VH-cDNA)
cag gtg cag ctg gtg cag tct gga gca gag gtg aaa aag ccc ggg gag
tct ctg aag atc tcc tgt ttg ggt tct gga ttc agc ttt acc aac tac
tgg atc ggc tgg gtg cgt cag atg ccc ggg aaa ggc ctg gag tcc atg
ggt atg atc ttt ctt ggt gac tct gat acc aga tac agc ccg tcc ttc
cga ggc cag gtc acc atc tca gcc gac aag tcc atc agt acc gcc tac
ctg cag tgg agc agc ctg agg gcc tcg gac acc gcc atg tat tac tgt
gcg cga ccc aaa gga ggc ggc tgg aac gac ttc tct ggt ttt gaa atc
tgg ggc caa ggg aca atg gtc acc gtc tct tca
SEQ ID NO: 16 (TRL10001 VH cDNA-CDR-H1--IMGT)
GGATTCAGCTTTACCAACTACTGG
SEQ ID NO: 17 (TRL10001 VH cDNA-CDR-H2--IMGT)
ATCTTTCTTGGTGACTCTGATACC
SEQ ID NO: 18 (TRL10001 VH CDNA-CDR-H3--IMGT)
GCGCGACCCAAAGGAGGCGGCTGGAACGACTTCTCTGGTTTTGAAATC
SEQ ID NO: 19 (TRL10001 VH cDNA-CDR-H1--KABAT)
AACTACTGGATCGGC
SEQ ID NO: 20 (TRL10001 VH cDNA-CDR-H2--KABAT)
ATGATCTTTCTTGGTGACTCTGATACCAGATACAGCCCGTCCTTCCGAGGC
SEQ ID NO: 21 (TRL10001 VH CDNA-CDR-H3--KABAT)
CCCAAAGGAGGCGGCTGGAACGACTTCTCTGGTTTTGAAATC
SEQ ID NO: 22 (TRL10001 VL Kappa-cDNA)
gat atc aca ctc acg cag tct cca act tcc ctg tct gca tct gta gga
gac aga gtc acc atc act tgc cgg gcg agt cag agc att aca aag ttt
tta aat tgg tat cag cag aaa cca ggg aaa gtc cct aag ctc ctg atc
aat gct gca tcc agt ttg caa agt ggg gtc cca tca agg ttc agt ggc
agt gga tct ggg aca gat tac act ctc acc atc agc aat ctc caa cct
gaa gat ttt gca act tac tac tgt caa cag agt ttc agc acc cct cgg
ccg ttc ggc caa ggg acg agg gtg gaa atc aaa cga
SEQ ID NO: 23 (TRL10001 VL CDNA-CDR-L1--IMGT)
CAGAGCATTACAAAGTTT
SEQ ID NO: 24 (TRL10001 VL CDNA--CDR-L2--IMGT)
GCTGCATCC
SEQ ID NO: 25 (TRL10001 VL CDNA--CDR-L3--IMGT)
CAACAGAGTTTCAGCACCCCTCGGCCG
SEQ ID NO: 26 (TRL10001 VL CDNA--CDR-L1--KABAT)
CGGGCGAGTCAGAGCATTACAAAGTTTTTAAAT
SEQ ID NO: 27 (TRL10001 VL CDNA--CDR-L2--KABAT)
GCTGCATCCAGTTTGCAAAGT
SEQ ID NO: 28 (TRL10001 VL CDNA-CDR-L3--KABAT)
CAACAGAGTTTCAGCACCCCTCGGCCG

What is claimed is:

1. An isolated monoclonal antibody (mAb) or antigen binding fragment thereof that specifically binds Anaplastic Lymphoma Kinase (ALK), the mAb or antigen binding fragment thereof comprising a heavy chain variable region (VH) as set forth in the amino acid sequence corresponding to SEQ ID NO:1 and a light chain variable (VL) region as set forth in the amino acid sequence corresponding SEQ ID NO: 8.

2. The isolated mAb or antigen binding fragment thereof of claim 1, wherein the mAb or antigen binding fragment thereof is monospecific, bispecific, or multispecific.

3. The isolated mAb or antigen binding fragment thereof of claim 1, wherein the mAb or antigen binding fragment thereof is a chimeric antibody or chimeric antigen binding fragment thereof, a human antibody or human antigen binding fragment thereof, a humanized antibody or humanized antigen binding fragment thereof, or a single chain antibody.

4. The isolated mAb or antigen binding fragment thereof of claim 1, wherein the mAb is a complete antibody.

5. The isolated mAb or antigen binding fragment thereof of claim 1, wherein the antigen binding fragment comprises a fragment selected from the group consisting of an Fv fragment, an Fab fragment, an Fab′ fragment, an F(ab′)₂ fragment, a single-chain Fv (scFV) fragment, and a single-chain Fab (scFab) fragment.

6.-9. (canceled)

10. A pharmaceutical or veterinary composition, the composition comprising the isolated mAb or antigen binding fragment thereof of claim 1.

11. An isolated nucleic acid that encodes, or a plurality of isolated nucleic acids that separately or in combination encodes, the variable light chain and the variable heavy chain regions of the mAb or antigen binding fragment thereof of claim 1.

12. A vector comprising the isolated nucleic acid or plurality of isolated nucleic acids of claim 11.

13. An isolated cell comprising the vector according to claim 12.

14. A method of targeting a cancer cell, comprising administering the mAb or antigen binding fragment of claim 1 to a subject.

15.-22. (canceled)

23. An isolated monoclonal antibody (mAb) or antigen binding fragment thereof that specifically binds Anaplastic Lymphoma Kinase (ALK), the isolated mAb or antigen binding fragment thereof comprising heavy chain variable region and a light chain variable region, wherein
(a) the heavy chain variable region comprises complementarity determining regions CDR-H1 (SEQ ID NO: 2), CDR-H2 (SEQ ID NO: 3), and CDR-H3 (SEQ ID NO: 4) and the light chain variable region comprises CDR-L1 (SEQ ID NO: 9), CDR-L2 (SEQ ID NO: 10), and CDR-L3 (SEQ ID NO: 11), or wherein
(b) the heavy chain variable region comprises complementarity determining regions CDR-H1 (SEQ ID NO: 5), CDR-H2 (SEQ ID NO: 6), and CDR-H3 (SEQ ID NO: 7) and the light chain variable region comprises CDR-L1 (SEQ ID NO: 12), CDR-L2 (SEQ ID NO: 13), and CDR-L3 (SEQ ID NO: 14).

24. (canceled)

25. The isolated mAb or antigen binding fragment thereof of claim 23, wherein the mAb or antigen binding fragment thereof is a chimeric antibody or chimeric antigen binding fragment thereof, a human antibody or human antigen binding fragment thereof, a humanized antibody or humanized antigen binding fragment thereof, or a single chain antibody.

26.-29. (canceled)

30. An immunoconjugate comprising (a) the isolated mAb or antigen binding fragment thereof of claim 23 and (b) an effector agent selected from the group comprising a toxin and a radioligand.

31.-32. (canceled)

33. An isolated nucleic acid that encodes, or a plurality of isolated nucleic acids that separately or in combination encode, the variable light chain and the variable heavy chain regions of the isolated mAb or antigen binding fragment thereof of claim 23.

34.-35. (canceled)

36. A method of treating cancer in a subject, the method comprising administering to the subject the isolated mAb or antigen binding fragment thereof of claim 23.

37. An isolated nucleic acid, or a plurality of isolated nucleic acids, wherein the isolated nucleic acid comprises, or the plurality of isolated nucleic acids comprise, a sequence that is 85%, 86%, 87%, 88%, 89%, 90%, 91%, 92%, 93%, 94%, 95%, 96%, 97%, 98%, 99%, or 100% identical to the nucleic acid sequence set forth in SEQ ID NOS: 15, 22, 30, 32, 34, 36, 38, 40, 42, or 44.

38. (canceled)

39. The isolated nucleic acid, or the plurality of isolated nucleic acids, of claim 37 wherein the isolated nucleic acid comprises, or the plurality of isolated nucleic acids comprise, the nucleic acid sequence set forth in SEQ ID NOS: 16, 17, and 18 or SEQ ID NOS: 23, 24, and 25.

40. A method of treating cancer, the method comprising administering to a subject one or more of the isolated nucleic acids set forth in claim 33.

41. A method of treating cancer, the method comprising administering to a subject any one or more nucleic acid sequences that encode the amino acid sequence set forth as SEQ ID NOS: 1-14, 29, 31, 33, 35, 37, 39, 41, or 43.

42. (canceled)

43. The method of claim 42, wherein nucleic acid is complexed with a lipid nanoparticle (LNP).


SEQUENCE LISTING (44 sequences)

SEQ ID NO: 1 - protein, 123 aa, Homo sapiens
  REGION 1..123: note = misc_feature - TRL10001 VH Region
QVQLVQSGAEVKKPGESLKISCLGSGFSFTNYWIGWVRQMPGKGLESMGMIFLGDSDTRYSPSFRGQVTISADKSISTAYLQWSSLRASDTAMYYCARPKGGGWNDFSGFEIWGQGTMVTVSS

SEQ ID NO: 2 - protein, 8 aa, Homo sapiens
  REGION 1..8: note = misc_feature - TRL10001 VH CDR-H1 -- IMGT
GFSFTNYW

SEQ ID NO: 3 - protein, 8 aa, Homo sapiens
  REGION 1..8: note = misc_feature - TRL10001 VH CDR-H2 -- IMGT
IFLGDSDT

SEQ ID NO: 4 - protein, 16 aa, Homo sapiens
  REGION 1..16: note = misc_feature - TRL10001 VH CDR-H3 -- IMGT
ARPKGGGWNDFSGFEI

SEQ ID NO: 5 - protein, 5 aa, Homo sapiens
  REGION 1..5: note = misc_feature - TRL10001 VH CDR1-H1 -- KABAT
NYWIG

SEQ ID NO: 6 - protein, 17 aa, Homo sapiens
  REGION 1..17: note = misc_feature - TRL10001 VH CDR-H2 -- KABAT
MIFLGDSDTRYSPSFRG

SEQ ID NO: 7 - protein, 14 aa, Homo sapiens
  REGION 1..14: note = misc_feature - TRL10001 VH CDR-H3 -- KABAT
PKGGGWNDFSGFEI

SEQ ID NO: 8 - protein, 108 aa, Homo sapiens
  REGION 1..108: note = misc_feature - TRL10001 VL (Kappa)
DITLTQSPTSLSASVGDRVTITCRASQSITKFLNWYQQKPGKVPKLLINAASSLQSGVPSRFSGSGSGTDYTLTISNLQPEDFATYYCQQSFSTPRPFGQGTRVEIKR

SEQ ID NO: 9 - protein, 6 aa, Homo sapiens
  REGION 1..6: note = misc_feature - TRL10001 VL CDR-L1 -- IMGT
QSITKF

SEQ ID NO: 10 - skipped

SEQ ID NO: 11 - protein, 9 aa, Homo sapiens
  REGION 1..9: note = misc_feature - TRL10001 VL CDR-L3 -- IMGT
QQSFSTPRP

SEQ ID NO: 12 - protein, 11 aa, Homo sapiens
  REGION 1..11: note = misc_feature - TRL10001 VL CDR-L1 -- KABAT
RASQSITKFLN

SEQ ID NO: 13 - protein, 7 aa, Homo sapiens
  REGION 1..7: note = misc_feature - TRL10001 VL CDR-L2 -- KABAT
AASSLQS

SEQ ID NO: 14 - protein, 9 aa, Homo sapiens
  REGION 1..9: note = misc_feature - TRL10001 VL CDR-L3 -- KABAT
QQSFSTPRP

SEQ ID NO: 15 - DNA, 369 bp, Homo sapiens
  misc_feature 1..369: note = TRL10001 VH - cDNA
  source 1..369: mol_type = genomic DNA
caggtgcagctggtgcagtctggagcagaggtgaaaaagcccggggagtctctgaagatctcctgtttgggttctggattcagctttaccaactactggatcggctgggtgcgtcagatgcccgggaaaggcctggagtccatgggtatgatctttcttggtgactctgataccagatacagcccgtccttccgaggccaggtcaccatctcagccgacaagtccatcagtaccgcctacctgcagtggagcagcctgagggcctcggacaccgccatgtattactgtgcgcgacccaaaggaggcggctggaacgacttctctggttttgaaatctggggccaagggacaatggtcaccgtctcttca

SEQ ID NO: 16 - DNA, 24 bp, Homo sapiens
  misc_feature 1..24: note = TRL10001 VH cDNA - CDR-H1 -- IMGT
  source 1..24: mol_type = genomic DNA
ggattcagctttaccaactactgg

SEQ ID NO: 17 - DNA, 24 bp, Homo sapiens
  misc_feature 1..24: note = TRL10001 VH cDNA - CDR-H2 -- IMGT
  source 1..24: mol_type = genomic DNA
atctttcttggtgactctgatacc

SEQ ID NO: 18 - DNA, 48 bp, Homo sapiens
  misc_feature 1..48: note = TRL10001 VH cDNA - CDR-H3 -- IMGT
  source 1..48: mol_type = genomic DNA
gcgcgacccaaaggaggcggctggaacgacttctctggttttgaaatc

SEQ ID NO: 19 - DNA, 15 bp, Homo sapiens
  misc_feature 1..15: note = TRL10001 VH cDNA - CDR-H1 -- KABAT
  source 1..15: mol_type = genomic DNA
aactactggatcggc

SEQ ID NO: 20 - DNA, 51 bp, Homo sapiens
  misc_feature 1..51: note = TRL10001 VH cDNA - CDR-H2 -- KABAT
  source 1..51: mol_type = genomic DNA
atgatctttcttggtgactctgataccagatacagcccgtccttccgaggc

SEQ ID NO: 21 - DNA, 42 bp, Homo sapiens
  misc_feature 1..42: note = TRL10001 VH cDNA - CDR-H3 -- KABAT
  source 1..42: mol_type = genomic DNA
cccaaaggaggcggctggaacgacttctctggttttgaaatc

SEQ ID NO: 22 - DNA, 324 bp, Homo sapiens
  misc_feature 1..324: note = TRL10001 VL Kappa - cDNA
  source 1..324: mol_type = genomic DNA
gatatcacactcacgcagtctccaacttccctgtctgcatctgtaggagacagagtcaccatcacttgccgggcgagtcagagcattacaaagtttttaaattggtatcagcagaaaccagggaaagtccctaagctcctgatcaatgctgcatccagtttgcaaagtggggtcccatcaaggttcagtggcagtggatctgggacagattacactctcaccatcagcaatctccaacctgaagattttgcaacttactactgtcaacagagtttcagcacccctcggccgttcggccaagggacgagggtggaaatcaaacga

SEQ ID NO: 23 - DNA, 18 bp, Homo sapiens
  misc_feature 1..18: note = TRL10001 VL cDNA - CDR-L1 -- IMGT
  source 1..18: mol_type = genomic DNA
cagagcattacaaagttt

SEQ ID NO: 24 - skipped

SEQ ID NO: 25 - DNA, 27 bp, Homo sapiens
  misc_feature 1..27: note = TRL10001 VL cDNA -- CDR-L3 -- IMGT
  source 1..27: mol_type = genomic DNA
caacagagtttcagcacccctcggccg

SEQ ID NO: 26 - DNA, 33 bp, Homo sapiens
  misc_feature 1..33: note = TRL10001 VL cDNA -- CDR-L1 -- KABAT
  source 1..33: mol_type = genomic DNA
cgggcgagtcagagcattacaaagtttttaaat

SEQ ID NO: 27 - DNA, 21 bp, Homo sapiens
  misc_feature 1..21: note = TRL10001 VL cDNA -- CDR-L2 -- KABAT
  source 1..21: mol_type = genomic DNA
gctgcatccagtttgcaaagt

SEQ ID NO: 28 - DNA, 27 bp, Homo sapiens
  misc_feature 1..27: note = TRL10001 VL cDNA - CDR-L3 -- KABAT
  source 1..27: mol_type = genomic DNA
caacagagtttcagcacccctcggccg

SEQ ID NO: 29 - protein, 122 aa, Homo sapiens
  REGION 1..122: note = misc_feature - TRL10005 VH
QVQLVESGGGLVKPGGSLRLSCAASGFTFNDHYMTWIRQAPGKGLEWISYISSSGGTIFYAESVKGRFTISRDNANNILFLQMNSLRAEDTAVYYCARGALRFLEWSHLGSWGQGTLVTVSS

SEQ ID NO: 30 - DNA, 366 bp, Homo sapiens
  misc_feature 1..366: note = TRL10005 VH cDNA
  source 1..366: mol_type = genomic DNA
caggtgcagctggtggagtccgggggaggcttggtcaagcctggagggtccctgagactctcctgtgcagcctctggattcaccttcaatgaccactacatgacctggatccgccaggctccagggaaggggctggaatggatttcatacattagtagtagtggtggtaccatcttctacgcagagtctgtgaagggccggttcaccatctccagggacaacgccaacaacatactgtttctgcagatgaacagcctgagagccgaggacacggccgtgtattattgtgcgagaggggcacttcgatttttggagtggtcccacctgggctcctggggccagggaaccctggtcaccgtctcctca

SEQ ID NO: 31 - protein, 109 aa, Homo sapiens
  REGION 1..109: note = misc_feature - TRL10005 VL (lambda)
DIVLTQSPGTLSLSPGERATLSCRASQRVSSSYLAWFQQKPGQAPRLLIYGASTRATGIPDRFSGSGSGTDFTLTISRLEPEDFAVYYCQQYGMSSWTFGQGTKVEIKR

SEQ ID NO: 32 - DNA, 327 bp, Homo sapiens
  misc_feature 1..327: note = TRL10005 VL (lambda) cDNA
  source 1..327: mol_type = genomic DNA
gatatcgtgttgacgcagtctccaggcaccctgtctttgtctccaggggaaagagccaccctctcctgcagggccagtcagagagttagtagcagctacttagcctggttccagcagaaacctggccaggctcccaggctcctcatctatggtgcatccaccagggccactggcatcccagacaggttcagtggcagcgggtctgggacagacttcactctaaccatcagcagactggagcctgaagattttgcagtgtattactgtcagcagtacggtatgtcatcgtggacgttcggccaagggaccaaggtggaaatcaaacga

SEQ ID NO: 33 - protein, 130 aa, Homo sapiens
  REGION 1..130: note = misc_feature - TRL10006 VH
QVQLLESGGGLIQPGVSLRLSCAASGFTFSNYNMNWVRQAPGKGLEWISYISSSSTTIYYADSVKGRFTISRDNAKNSLYLQLDSLRAEDTAVYYCARGFRPDIFIGVHGLSYHYGMDVWGQGTTVTVSS

SEQ ID NO: 34 - DNA, 390 bp, Homo sapiens
  misc_feature 1..390: note = TRL10006 VH cDNA
  source 1..390: mol_type = genomic DNA
caggtgcagctgctcgagtcggggggaggcttgatacagcctggggtgtccctgagactctcctgtgcagcctctgggttcaccttcagtaactataacatgaactgggtccgccaggctccagggaaggggctggagtggatttcatacattagtagtagtagtactaccatatactacgcagactctgtgaagggccgattcaccatctccagagacaacgccaagaattcactgtatctgcaattggacagcctgagagccgaggacacggctgtatattactgtgcgagaggatttcgccccgatattttcattggtgttcacggtctaagctatcactacggcatggacgtctggggccaagggaccacggtcaccgtctcctca

SEQ ID NO: 35 - protein, 110 aa, Homo sapiens
  REGION 1..110: note = misc_feature - TRL10006 VL (lambda)
DIQMTQSPSVSAAPGQRVTISCSGSSSNIGNRDVSWYQQLPGTAPKLLIYDNNKRPSGIPDRFSGSKSGTSATLGITGLQTGDEADYYCGTWDNSLTSWVFGGGTKLTVL

SEQ ID NO: 36 - DNA, 333 bp, Homo sapiens
  misc_feature 1..333: note = TRL10006 VL (lambda) cDNA
  source 1..333: mol_type = genomic DNA
gatatccagatgacccagtctccctcagtgtctgcggccccaggacagagggtcaccatctcctgctctggaagcagctccaacattgggaatagagatgtatcctggtaccagcagctcccaggaacagcccccaaactcctgatttatgacaataataagcgaccctcagggattcctgaccgattctctggctccaagtctggcacgtcagccaccctgggcatcaccggactccagacaggggacgaggccgattattactgcggaacatgggataacagcctgacttcctgggttttcggcggagggaccaagctgaccgtcctggtt

SEQ ID NO: 37 - protein, 122 aa, Homo sapiens
  REGION 1..122: note = misc_feature - TRL10014 VH
QVQLVESGGGLVKPGGSLRLSCTASGFSFSDSYMNWIRQAPGKGLEWLAYISTSATYTYSADSVKGRFTISRDNAKNSLYLQMNSLRDEDTAVYYCAREGLFYDGLYGMDVWGQGTTVIVSS

SEQ ID NO: 38 - DNA, 366 bp, Homo sapiens
  misc_feature 1..366: note = TRL10014 VH cDNA
  source 1..366: mol_type = genomic DNA
caggtgcagctggtggagtctgggggaggcttggtcaagcctggagggtccctgagactctcctgtacagcctctggattctccttcagtgactcctacatgaactggatccgccaggctccaggtaaggggctggagtggcttgcatacattagtaccagtgctacttacacatactccgcagactctgtgaagggccgattcaccatctccagagacaacgccaagaactcactgtatctacaaatgaacagcctgagagacgaggacacggccgtctattattgtgcgagagaggggctcttctacgatgggctctacggtatggacgtctggggccaagggaccacggtcatcgtctcctca

SEQ ID NO: 39 - protein, 113 aa, Homo sapiens
  REGION 1..113: note = misc_feature - TRL10014 VL (kappa)
DILMTQSPLSLPVTPGEPASISCRSSQSLLHSNGYNYLNWYLQKPGQSPQLLIYLGSNRASGVPDRFSGSGSGTDFTLKISSVEAEDVGVYYCMQALHTPRTFGQGTKVEIKR

SEQ ID NO: 40 - DNA, 339 bp, Homo sapiens
  misc_feature 1..339: note = TRL10014 VL (kappa) cDNA
  source 1..339: mol_type = genomic DNA
gatatcctgatgacccagtctccactctccctgcccgtcacccctggagagccggcctccatctcctgcaggtctagtcagagcctcctgcatagtaatggatacaactatttgaattggtacctgcagaagccagggcagtctccacagctcctgatctatttgggttctaatcgggcctccggggtccctgacaggttcagcggcagtggatcaggcacagattttacactgaaaataagcagcgtggaggctgaggatgttggggtttattactgcatgcaagctctacacactcctcggacgttcggccaagggaccaaggtggaaatcaaacga

SEQ ID NO: 41 - protein, 125 aa, Homo sapiens
  REGION 1..125: note = misc_feature - TRL10051 VH
QVQLVESGGGLVQPGGSLRLSCAASGFTFSSYVMSWVRQAPGKGLEWVSSIRVSGDRAHYADSVKGRFTVSRDDSKNTVYLQMNSLRVEDTAVYYCVKDQAERLLRAVTTHFDYWGQGALVTVSS

SEQ ID NO: 42 - DNA, 375 bp, Homo sapiens
  misc_feature 1..375: note = TRL10051 VH cDNA
  source 1..375: mol_type = genomic DNA
caggtgcagctggtggagtctgggggaggcttggtccagccgggggggtccctgagactctcctgtgcagcctctggattcaccttcagcagctatgtcatgagctgggtccgccaggctccagggaaggggctggagtgggtctcaagtattagagttagtggtgatcgcgcacactacgcagactccgtgaaggggcggttcaccgtctccagagacgattccaagaacacggtgtatctgcaaatgaacagcctgagagtcgaagacacggccgtatattactgtgtgaaagatcaagccgaacgtctccttagggcggtgactacacattttgactactggggccagggagccctcgtcaccgtctcctca

SEQ ID NO: 43 - protein, 113 aa, Homo sapiens
  REGION 1..113: note = misc_feature - TRL10051 VL (kappa)
DIVMTQSPLSSPVTLGQPASISCRSSQSLVHSDGNTYLSWLQQRPGQPPRLLIYKVSRRFSGVPDRFSGSGAGTDFTLKISRVEAEDVGIYYCMQASQFPRTFGQGTKVEIKR

SEQ ID NO: 44 - DNA, 339 bp, Homo sapiens
  misc_feature 1..339: note = TRL10051 VL (kappa) cDNA
  source 1..339: mol_type = genomic DNA
gatatcgtgatgacccagtctccactctcctcacctgtcacccttggacagccggcctccatttcctgcaggtctagtcaaagcctcgtacacagtgatggaaacacctacttgagttggcttcagcagaggccaggccagcctccaagactcctaatttataaggtttctaggcggttctctggggtcccagacagattcagtggcagtggggcaggcacagatttcacactgaaaatcagcagggtggaagctgaggatgtcgggatttattactgcatgcaagcttcacaatttccgcgaacgttcggccaagggaccaaggtggaaatcaaacga